(19) United States
(12) Patent Application Publication
(10) Pub. No.: US 2025/0263423 A1
(43) Pub. Date: Aug. 21, 2025

(54) INHIBITORS OF RAS ONCOPROTEINS

(71) Applicant: BridGene Biosciences, Inc., San Jose, CA (US)

(72) Inventors: Chao ZHANG, Monterey Park, CA (US); Henry William Beecroft JOHNSON, Woodside, CA (US); Ping CAO, Palo Alto, CA (US)

(21) Appl. No.: 18/849,907

(22) PCT Filed: Dec. 21, 2022
(86) PCT No.: PCT/US2022/053613

§ 371 Date: Sep. 23, 2024

Related U.S. Application Data
(60) Provisional application No. 63/292,416, filed on Dec. 21, 2021.

(51) Int. Cl. C07D 519/00; A61K 31/519; A61K 31/55

(52) U.S. Cl. CPC C07D 519/00; A61K 31/519; A61K 31/55

(57) ABSTRACT

A class of compounds useful in cancer therapy having formula (I): or an optically pure stereoisomer, pharmaceutically acceptable salt, or solvate thereof, wherein X is a nitrogen or an unsubstituted C₁ group; Y is a nitrogen, a substituted or an unsubstituted C₁ group; Z is a nitrogen, a substituted or an unsubstituted C₁ group; R¹ is selected from the group consisting of: wherein R⁴ is independently in each instance a bond, a C₁₋₂ alkyl group; R⁵ is independently in each instance a hydrogen or a C₁ alkyl group; D is a hydrogen or a C₁ alkyl; E is a NH, oxygen, carbonyl, substituted or unsubstituted C₁ alkyl; R² is selected from the group consisting of: and; wherein R⁶ is selected from the group consisting of a hydrogen, a halogen, an alkyl group; and R³ is a substituted or unsubstituted C₆₋₁₈ aryl group.

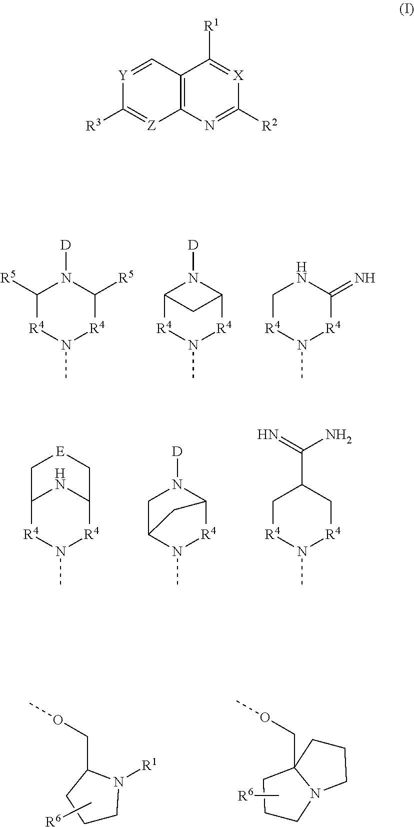

### CROSS-REFERENCE TO RELATED APPLICATIONS

The application claims benefit of priority under 35 U.S.C. § 119(e) of U.S. Ser. No. 63/292,416, filed Dec. 21, 2021, the entire content of which is incorporated by reference.

### FIELD

The present disclosure relates generally to the field of compounds, pharmaceutical compositions, and methods of using the compounds and compositions containing them. The present disclosure specifically relates to GTPase inhibitor compounds and compositions containing them, and the use of the compounds and compositions for the treatment of cancer.

### BACKGROUND OF THE INVENTION

The present invention relates generally to cancer therapy, and more specifically to the synthesis of inhibitors of RAS oncoproteins and use thereof for the treatment of cancers.

RAS (Rat sarcoma viral oncogene homologue) proteins are a group of GTP-binding proteins which regulate cellular processes, such as migration, differentiation, and proliferation. This class of proteins cycle between the inactive state (GDP-bound) and active state (GTP-bound), a process which relays signals in response to extracellular stimuli. This class of proteins are termed GTPases because of their ability to hydrolyze GTP to GDP and phosphate as enzymes. Oncogenic mutation of one such family member, KRAS, is associated with tumorigensis and is regarded as the most frequently mutated oncogene. Patients with KRAS mutations are often resistant to standard-of-care therapy and have a poor prognosis. Accordingly, inhibitors of mutant KRAS can have great potential in the application of cancer therapy. See Merz, V.; Gaule, M.; Zecchetto, C.; Cavaliere, A.; Casalino, S.; Pesoni, C.; Contarelli, S.; Sabbadini, F. et al. Front. Oncol. 2021, 11, 638360.

### SUMMARY OF THE INVENTION

The present invention is based on the seminal discovery of a class of compounds effective as inhibitors of RAS oncoproteins. These compounds can be used in cancer therapy.

The compounds of the present invention are as follows:

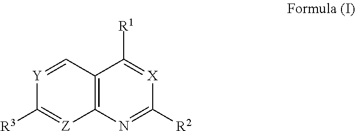

or an optically pure stereoisomer, pharmaceutically acceptable salt, or solvate thereof,
wherein
X is a nitrogen or an unsubstituted C₁ group;
Y is a nitrogen, a substituted or an unsubstituted C₁ group;
Z is a nitrogen, a substituted or an unsubstituted C₁ group;
R¹ is selected from the group consisting of:

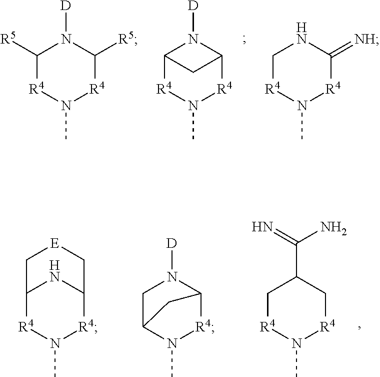

wherein R⁴ is independently in each instance a bond, a C₁₋₂ alkyl group;
R⁵ is independently in each instance a hydrogen or a C₁ alkyl group;
D is a hydrogen or a C₁ alkyl;
E is a NH, oxygen, carbonyl, substituted or an unsubstituted C₁ alkyl;
R² is selected from the group consisting of:

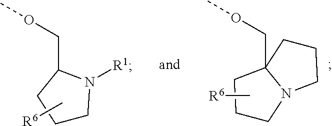

wherein R⁶ is selected from the group consisting of a hydrogen, a halogen, an alkyl group; and
R³ is a substituted or an unsubstituted C₆₋₁₈ aryl group.

In some specific embodiments, E is selected from the group consisting of NH, O, CH₂, CHOH, CH₂F, CHF₂, and C═O. In one embodiment, R⁶ is a C₁₋₅ alkyl group. In yet other specific embodiments, R³ is a C₅₋₆ aryl group, a C₁₀₋₁₂ biaryl group, or a C₅₋₆ heteroaryl group, wherein they may further be substituted with alkyl, —OMe, —F, —Cl, —OMe alone or with di- or tri-substitutions. Further, Y and Z may independently at each instance may be a substituted C₁ alkyl group, and may include CF, and CCl groups.

In some embodiments, the compound of the invention has formula X:

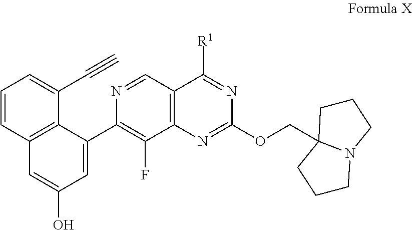

wherein R¹ is selected from the group consisting of:

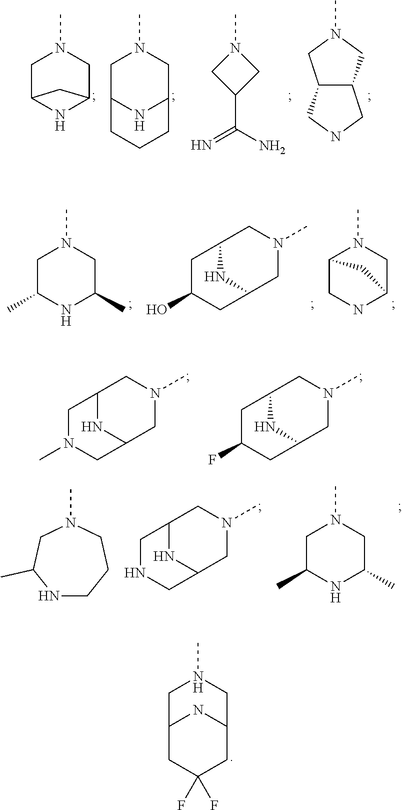

Also disclosed herein is a pharmaceutical composition including a compound according to Formula (I).

Further disclosed herein is a method for treating cancer in a subject including administering a compound of Formula (I). In some embodiments, the cancer may be selected from the group consisting of breast, lung, bladder, prostate, ovarian, endometrial, rhabdomyosarcoma, liver, gastric, colon/colorectal, and pancreatic cancers. In some embodiments, the method also includes administering a chemotherapeutic agent, the compound can be administered prior to, simultaneously with or following the administration of the chemotherapeutic agent.

Also disclosed herein is a method of inhibiting a GTPase activity including contacting a cell with a compound of Formula (I).

Other features and advantages can become apparent from the following detailed description.

### DETAILED DESCRIPTION OF PREFERRED EMBODIMENTS OF THE INVENTION

Below are some acronyms used in the present disclosure. t-BuOK refers to potassium tert-butoxide; DMF refers to dimethylformamide; Boc refers to tert-Butyloxycarbonyl protecting group; DMSO refers to dimethyl sulfoxide; HATU refers to 1-[bis(dimethylamino)methylene]-1H-1,2,3-triazolo[4,5-b]pyridinium 3-oxide hexafluorophosphate; DIEA refers to N, N-Diisopropylethylamine; DIPEA refers to N,N-Diisopropylethylamine; TFA refers to trifluoroacetic acid; OMOM refers to methoxy methyl ether.

The term “about” will be understood by persons of ordinary skill in the art. Whether the term “about” is used explicitly or not, every quantity given herein refers to the actual given value, and it is also meant to refer to the approximation to such given value that would be reasonably inferred based on the ordinary skill in the art.

Alkyl groups refer to univalent groups derived from alkanes by removal of a hydrogen atom from any carbon atom, which include straight chain and branched chain with from 1 to 12 carbon atoms, and typically from 1 to about 10 carbons or in some embodiments, from 1 to about 6 carbon atoms, or in other embodiments having 1, 2, 3 or 4 carbon atoms. Examples of straight chain alkyl groups include, but are not limited to, methyl, ethyl, n-propyl, n-butyl, n-pentyl, and n-hexyl groups. Examples of branched chain alkyl groups include, but are not limited to isopropyl, isobutyl, sec-butyl and tert-butyl groups.

Alkyl groups may be substituted or unsubstituted. Representative substituted alkyl groups may be mono-substituted or substituted more than once, such as, but not limited to, di-, or tri-substituted. As used herein, the term alkyl, unless otherwise stated, refers to both cyclic and noncyclic groups.

The terms “cyclic alkyl” or “cycloalkyl” refer to univalent groups derived from cycloalkanes by removal of a hydrogen atom from a ring carbon atom. Cycloalkyl groups are saturated or partially saturated non-aromatic structures with a single ring or multiple rings including isolated, fused, bridged, and spiro ring systems, having 3 to 14 carbon atoms, or in some embodiments, from 3 to 12, or 3 to 10, or 3 to 8, or 3, 4, 5, 6 or 7 carbon atoms. Cycloalkyl groups may be substituted or unsubstituted. Representative substituted cycloalkyl groups may be mono-substituted or substituted more than once, such as, but not limited to, di- or tri-substituted. Examples of monocyclic cycloalkyl groups include, but are not limited to cyclopropyl, cyclobutyl, cyclopentyl, and cyclohexyl groups. Examples of multi-cyclic ring systems include, but are not limited to, bicycle[4.4.0]decane, bicycle[2.2.1]heptane, spiro[2.2]pentane, and the like.

Alkenyl groups refer to straight and branched chain and cycloalkyl groups as defined above, with one or more double bonds between two carbon atoms. Alkenyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkenyl groups may be substituted or unsubstituted. Representative substituted alkenyl groups may be mono-substituted or substituted more than once, such as, but not limited to, di-, or tri-substituted. Examples of alkenyl groups include, but are not limited to, vinyl, allyl, —CH═CH(CH₃), —CH═C(CH₃)₂, —C(CH₃)═CH₂, cyclopentenyl, cyclohexenyl, butadienyl, pentadienyl, and hexadienyl, among others.

Alkynyl groups refer to straight and branched chain and cycloalkyl groups as defined above, with one or more triple bonds between two carbon atoms. Alkynyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkynyl groups may be substituted or unsubstituted. Representative substituted alkynyl groups may be mono-substituted or substituted more than once, such as, but not limited to, mono-, di-, or tri-substituted. Exemplary alkynyl groups include, but are not limited to, ethynyl, propargyl, and —C≡C(CH₃), among others.

Aryl groups are cyclic aromatic hydrocarbons that include single and multiple ring compounds, including multiple ring compounds that contain separate and/or fused aryl groups. Aryl groups may contain from 6 to about 18 ring carbons, or in some embodiments from 6 to 14 ring carbons or even 6 to 10 ring carbons in other embodiments. Aryl group also includes heteroaryl groups, which are aromatic ring compounds containing 5 or more ring members, one or more ring carbon atoms of which are replaced with heteroatom such as, but not limited to, N, O, and S. Aryl groups may be substituted or unsubstituted. Representative substituted aryl groups may be mono-substituted or substituted more than once, such as, but not limited to, di-, or tri-substituted. Aryl groups include, but are not limited to, phenyl, biphenylenyl, triphenylenyl, naphthyl, anthryl, and pyrenyl groups.

Suitable heterocyclyl groups include cyclic groups with atoms of at least two different elements as members of its rings, of which one or more is a heteroatom such as, but not limited to, N, O, or S. Heterocyclyl groups may include 3 to about 20 ring members, or 3 to 18 in some embodiments, or about 3 to 15, 3 to 12, 3 to 10, or 3 to 6 ring members. The ring systems in heterocyclyl groups may be unsaturated, partially saturated, and/or saturated. Heterocyclyl groups may be substituted or unsubstituted. Representative substituted heterocyclyl groups may be mono-substituted or substituted more than once, such as, but not limited to, di-, or tri-substituted. Exemplary heterocyclyl groups include, but are not limited to, pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, aziridinyl, imidazolidinyl, pyrazolidinyl, thiazolidinyl, tetrahydrothiophenyl, tetrahydrofuranyl, dioxolyl, furanyl, thiophenyl, pyrrolyl, imidazolyl, pyrazolyl, pyrazolinyl, triazolyl, tetrazolyl, oxazolyl, isoxazolyl, thiazolyl, thiazolinyl, oxetanyl, thietanyl, homopiperidyl, oxepanyl, thiepanyl, oxazepinyl, diazepinyl, thiazepinyl, 1,2,3,6-tetrahydropyridyl, indolinyl, 2H-pyranyl, 4H-pyranyl, dioxolanyl, dioxanyl, purinyl, quinolizinyl, cinnolinyl, phthalazinyl, pteridinyl, and benzothiazolyl groups.

Polycyclic or polycyclyl groups refer to two or more rings in which two or more carbons are common to the two adjoining rings, wherein the rings are “fused rings”; if the rings are joined by one common carbon atom, these are “spiro” ring systems. Rings that are joined through non-adjacent atoms are “bridged” rings. Polycyclic groups may be substituted or unsubstituted. Representative polycyclic groups may be substituted one or more times.

Halogen groups include F, Cl, Br, and I; nitro group refers to —NO₂; cyano group refers to —CN; isocyano group refers to —N—C; epoxy groups encompass structures in which an oxygen atom is directly attached to two adjacent or non-adjacent carbon atoms of a carbon chain or ring system, which is essentially a cyclic ether structure. An epoxide is a cyclic ether with a three-atom ring.

An alkoxy group is a substituted or unsubstituted alkyl group, as defined above, singular bonded to oxygen. Alkoxy groups may be substituted or unsubstituted. Representative substituted alkoxy groups may be substituted one or more times. Exemplary alkoxy groups include, but are not limited to, methoxy, ethoxy, propoxy, butoxy, pentoxy, hexoxy, isopropoxy, sec-butoxy, tert-butoxy, cyclopropyloxy, cyclobutyloxy, cyclopentyloxy, and cyclohexyloxy groups.

The terms “amine” and “amino” refer to derivatives of ammonia, wherein one of more hydrogen atoms have been replaced by a substituent which include, but are not limited to alkyl, alkenyl, aryl, and heterocyclyl groups. Carbamate groups refers to —O(C═O)NR₁R₂, where R₁ and R₂ are independently hydrogen, aliphatic groups, aryl groups, or heterocyclyl groups.

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those needing preventive measures).

The term “subject” as used herein refers to any individual or patient to which the subject methods are performed. Generally, the subject is human, although as will be appreciated by those in the art, the subject may be an animal.

The terms “therapeutically effective amount”, “effective dose”, “therapeutically effective dose”, “effective amount,” or the like refer to the amount of a subject compound that will elicit the biological or medical response in a tissue, system, animal or human that is being sought by administering said compound. Generally, the response is either amelioration of symptoms in a patient or a desired biological outcome. Such amount should be sufficient to inhibit GTPase enzymatic activity.

Also disclosed herein are pharmaceutical compositions including compounds with the structures of Formula (I), Formula (II), Formula (III), Formula (IV), Formula (V), Formula (VI), Formula (VII), Formula (VIII), Formula (IX), or Formula (X). The term “pharmaceutically acceptable carrier” refers to a non-toxic carrier that may be administered to a patient, together with a compound of this disclosure, and which does not destroy the pharmacological activity thereof. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable carriers that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

In pharmaceutical composition comprising only the compounds described herein as the active component, methods for administering these compositions may additionally comprise the step of administering to the subject an additional agent or therapy. Such therapies include, but are not limited to, an anemia therapy, a diabetes therapy, a hypertension therapy, a cholesterol therapy, neuropharmacologic drugs, drugs modulating cardiovascular function, drugs modulating inflammation, immune function, production of blood cells; hormones and antagonists, drugs affecting gastrointestinal function, chemotherapeutics of microbial diseases, and/or chemotherapeutics of neoplastic disease. Other pharmacological therapies can include any other drug or biologic found in any drug class. For example, other drug classes can comprise allergy/cold/ENT therapies, analgesics, anesthetics, anti-inflammatories, antimicrobials, antivirals, asthma/pulmonary therapies, cardiovascular therapies, dermatology therapies, endocrine/metabolic therapies, gastrointestinal therapies, cancer therapies, immunology therapies, neurologic therapies, ophthalmic therapies, psychiatric therapies or rheumatologic therapies. Other examples of agents or therapies that can be administered with the compounds described herein include a matrix metalloprotease inhibitor, a lipoxygenase inhibitor, a cytokine antagonist, an immunosuppressant, a cytokine, a growth factor, an immunomodulator, a prostaglandin or an anti-vascular hyperproliferation compound.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology).

The compounds of this disclosure may be employed in a conventional manner for controlling the disease described herein, including, but not limited to, cancer. Such methods of treatment, their dosage levels and requirements may be selected by those of ordinary skill in the art from available methods and techniques. For example, the compounds of this disclosure may be combined with a pharmaceutically acceptable adjuvant for administration to a patient suffering from cancer in a pharmaceutically acceptable manner and in an amount effective to treat cancer.

Alternatively, the compounds of this disclosure may be used in compositions and methods for treating or protecting individuals against the diseases described herein, including but not limited to a cancer, over extended periods of time. The compounds may be employed in such compositions either alone or together with other compounds of this disclosure in a manner consistent with the conventional utilization of such compounds in pharmaceutical compositions. For example, a compound of this disclosure may be combined with pharmaceutically acceptable adjuvants conventionally employed in vaccines and administered in prophylactically effective amounts to protect individuals over an extended period of time against the diseases described herein, including, but not limited to, cancer.

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

When the compounds of this disclosure are administered in combination therapies with other agents, they may be administered sequentially or concurrently to the patient. Alternatively, pharmaceutical or prophylactic compositions according to this disclosure comprise a combination of ivermectin, or any other compound described herein, and another therapeutic or prophylactic agent. Additional therapeutic agents that are normally administered to treat a particular disease or condition may be referred to as “agents appropriate for the disease, or condition, being treated.”

The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

According to a preferred embodiment, the compositions of this disclosure are formulated for pharmaceutical administration to a subject or patient, e.g., a mammal, preferably a human being. Such pharmaceutical compositions are used to ameliorate, treat or prevent any of the diseases described herein including but not limited to cancer in a subject.

Agents of the disclosure are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present disclosure provides pharmaceutically acceptable compositions comprising a therapeutically effective amount of one or more of a described compound, formulated together with one or more pharmaceutically acceptable carriers (additives) and/or diluents for use in treating the diseases described herein, including, but not limited to cancer. While it is possible for a described compound to be administered alone, it is preferable to administer a described compound as a pharmaceutical formulation (composition) as described herein. Described compounds may be formulated for administration in any convenient way for use in human or veterinary medicine, by analogy with other pharmaceuticals.

As described in detail, pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient, which can be combined with a carrier material, to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient. In some embodiments, this amount will range from about 5% to about 70%, from about 10% to about 50%, or from about 20% to about 40%.

In certain embodiments, a formulation as described herein comprises an excipient selected from the group consisting of cyclodextrins, liposomes, micelle forming agents, e.g., bile acids, and polymeric carriers, e.g., polyesters and polyanhydrides; and a compound of the present disclosure. In certain embodiments, an aforementioned formulation renders orally bioavailable a described compound of the present disclosure.

Methods of preparing formulations or compositions comprising described compounds include a step of bringing into association a compound of the present disclosure with the carrier and, optionally, one or more accessory ingredients. In general, formulations may be prepared by uniformly and intimately bringing into association a compound of the present disclosure with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof, and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this disclosure may also be administered in the form of suppositories for rectal administration. These compositions can be prepared by mixing a compound of this disclosure with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically-administered transdermal patches are also included in this disclosure.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Transdermal patches have the added advantage of providing controlled delivery of a compound of the present disclosure to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/orantifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In certain embodiments, a described compound or pharmaceutical preparation is administered orally. In other embodiments, a described compound or pharmaceutical preparation is administered intravenously. Alternative routes of administration include sublingual, intramuscular, and transdermal administrations.

When compounds described herein are administered as pharmaceuticals, to humans and animals, they can be given per se or as a pharmaceutical composition containing, for example, 0.1% to 99.5% of active ingredient in combination with a pharmaceutically acceptable carrier. In some embodiments, 0.5% to 90% of active ingredient can be used.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, compounds described herein which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of the disclosure may be varied so as to obtain an amount of the active ingredient that is effective to achieve the desired therapeutic response for a particular patient, composition, and mode of administration, without being toxic to the patient.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization.

The term “cancer” refers to a group diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

Exemplary cancers include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood′, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland′Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In certain aspects, cancer includes Lung cancer, Breast cancer, Colorectal cancer, Prostate cancer, Stomach cancer, Liver cancer, cervical cancer, Esophageal cancer, Bladder cancer, Non-Hodgkin lymphoma, Leukemia, Pancreatic cancer, Kidney cancer, endometrial cancer, Head and neck cancer, Lip cancer, oral cancer, Thyroid cancer, Brain cancer, Ovary cancer, Melanoma, Gallbladder cancer, Laryngeal cancer, Multiple myeloma, Nasopharyngeal cancer, Hodgkin lymphoma, Testis cancer and Kaposi sarcoma.

In certain aspects, the method further includes administering a chemotherapeutic agent. The compounds of the disclosure can be administered in combination with one or more additional therapeutic agents. The phrases “combination therapy”, “combined with” and the like refer to the use of more than one medication or treatment simultaneously to increase the response. The GTPase inhibitor of the present disclosure might for example be used in combination with other drugs or treatment in use to treat cancer. In various aspect, the compound is administered prior to, simultaneously with or following the administration of the chemotherapeutic agent.

The term “anti-cancer therapy” refers to any therapy or treatment that can be used for the treatment of a cancer. Anti-cancer therapies include, but are not limited to, surgery, radiotherapy, chemotherapy, immune therapy and targeted therapies.

Examples of chemotherapeutic agents or anti-cancer agents include, but are not limited to, Actinomycin, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fiuorouracil, Gemcitabine, Hydroxyurea, Idarubicin, Imatinib, lrinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, Vinorelbine, panitumamab, Erbitux (cetuximab), matuzumab, IMC-IIF 8, TheraCIM hR3, denosumab, Avastin (bevacizumab), Humira (adalimumab), Herceptin (trastuzumab), Remicade (infliximab), rituximab, Synagis (palivizumab), Mylotarg (gemtuzumab oxogamicin), Raptiva (efalizumab), Tysabri (natalizumab), Zenapax (dacliximab), NeutroSpec (Technetium (99mTc) fanolesomab), tocilizumab, ProstaScint (Indium-Ill labeled Capromab Pendetide), Bexxar (tositumomab), Zevalin (ibritumomab tiuxetan (IDEC-Y2B8) conjugated to yttrium 90), Xolair (omalizumab), MabThera (Rituximab), ReoPro (abciximab), MabCampath (alemtuzumab), Simulect (basiliximab), LeukoScan (sulesomab), CEA-Scan (arcitumomab), Verluma (nofetumomab), Panorex (Edrecolomab), alemtuzumab, CDP 870, natalizumab Gilotrif (afatinib), Lynparza (olaparib), Perjeta (pertuzumab), Otdivo (nivolumab), Bosulif (bosutinib), Cabometyx (cabozantinib), Ogivri (trastuzumab-dkst), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib). Examples of immunotherapeutic agent include, but are not limited to, interleukins (Il-2, Il-7, Il-12), cytokines (Interferons, G-CSF, imiquimod), chemokines (CCL3, CC126, CXCL7), immunomodulatory imide drugs (thalidomide and its analogues).

In treatment, the dose of agent optionally ranges from about 0.0001 mg/kg to about 100 mg/kg, about 0.01 mg/kg to about 5 mg/kg, about 0.15 mg/kg to about 3 mg/kg, 0.5 mg/kg to about 2 mg/kg and about 1 mg/kg to about 2 mg/kg of the subject's body weight. In other embodiments the dose ranges from about 100 mg/kg to about 5 g/kg, about 500 mg/kg to about 2 mg/kg and about 750 mg/kg to about 1.5 g/kg of the subject's body weight. For example, depending on the type and severity of the disease, about 1 μg/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

In some embodiments, an agent is administered to a human patient at an effective amount (or dose) of less than about 1 μg/kg, for instance, about 0.35 to about 0.75 μg/kg or about 0.40 to about 0.60 μg/kg. In some embodiments, the dose of an agent is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various embodiments, the absolute dose of an agent is about 2 μg/subject to about 45 μg/subject, or about 5 to about 40, or about 10 to about 30, or about 15 to about 25 μg/subject. In some embodiments, the absolute dose of an agent is about 20 μg, or about 30 μg, or about 40 μg.

In various embodiments, the dose of an agent may be determined by the human patient's body weight. For example, an absolute dose of an agent of about 2 μg for a pediatric human patient of about 0 to about 5 kg (e.g. about 0, or about 1, or about 2, or about 3, or about 4, or about 5 kg); or about 3 μg for a pediatric human patient of about 6 to about 8 kg (e.g. about 6, or about 7, or about 8 kg), or about 5 μg for a pediatric human patient of about 9 to about 13 kg (e.g. 9, or about 10, or about 11, or about 12, or about 13 kg); or about 8 μg for a pediatric human patient of about 14 to about 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 μg for a pediatric human patient of about 31 to about 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to about 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

In certain embodiments, an agent in accordance with the methods provided herein is administered subcutaneously (s.c.), intraveneously (i.v.), intramuscularly (i.m.), intranasally or topically. Administration of an agent described herein can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, an agent is administered about 1 to about 3 times (e.g. 1, or 2 or 3 times).

Presented below are examples discussing the design and evaluation of efficacy of new GTPase inhibitors, contemplated for the discussed applications. The following examples are provided to further illustrate the embodiments of the present disclosure, but are not intended to limit the scope of the disclosure. While they are typical of those that might be used, other procedures, methodologies, or techniques known to those skilled in the art may alternatively be used.

The following examples are presented to illustrate, but not to limit, the present invention.

### Example 1

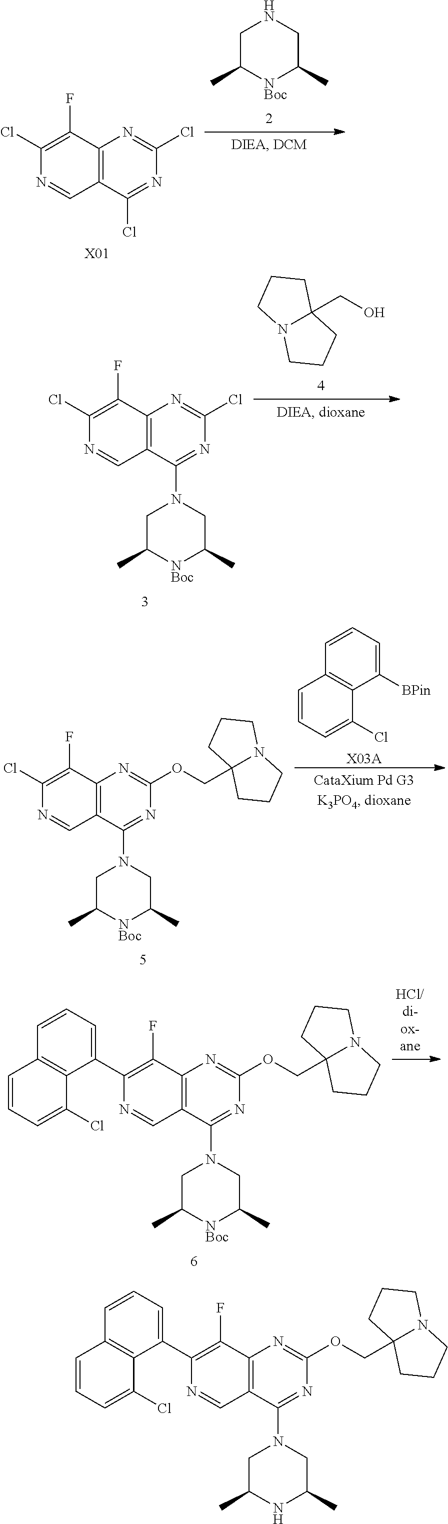

### Procedure for Preparation of Compound 3

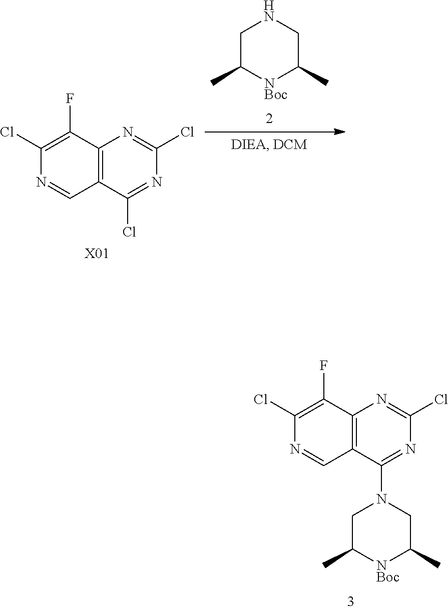

To a mixture of tert-butyl (2S, 6R)-2,6-dimethylpiperazine-1-carboxylate (361 mg, 1.68 mmol) in DCM (10 mL) and DIEA (2.56 g, 19.81 mmol) was added 2,4,7-trichloro-8-fluoro-pyrido[4,3-d]pyrimidine (500 mg, 1.98 mmol) at −40° C. The mixture was stirred at −40° C. for 0.5 h then concentrated to dryness. The mixture was partitioned between H₂O (60 mL) and ethyl acetate (60 mL). The organic phase was separated and the aqueous phase was extracted with ethyl acetate (60 mL×2). The combined organic layers were washed with brine (60 mL), dried over Na₂SO₄, filtered and concentrated under vacuum. The residue was purified by flash column chromatography on silica gel (MeOH in DCM from 0% to 10%) to afford tert-butyl (2S, 6R)-4-(2,7-dichloro-8-fluoro-pyrido[4,3-d]pyrimidin-4-yl)-2,6-dimethyl-piperazine-1-carboxylate (667 mg, 74% yield) as a yellow solid.

### Procedure for Preparation of Compound 5

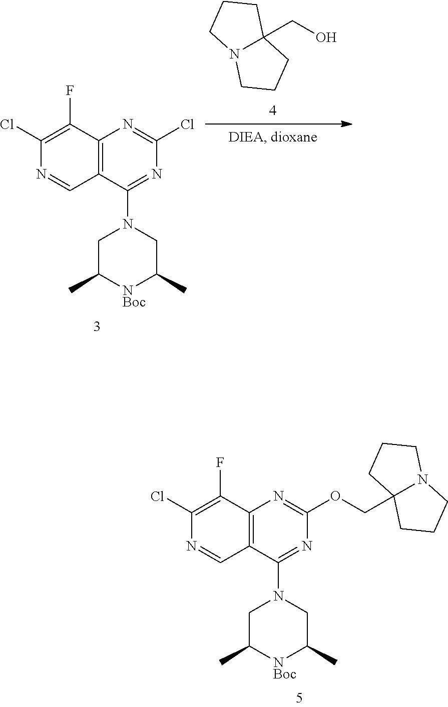 [SMILES: OCC12CCCN1CCC2 ; C[C@@H]1CN(c2nc(Cl)nc3c(F)c(Cl)ncc23)C[C@H](C)N1C(=O)OC(C)(C)C ; C[C@@H]1CN(c2nc(OCC34CCCN3CCC4)nc3c(F)c(Cl)ncc23)C[C@H](C)N1C(=O)OC(C)(C)C]

A mixture of tert-butyl (2S, 6R)-4-(2,7-dichloro-8-fluoro-pyrido[4,3-d]pyrimidin-4-yl)-2,6-dimethyl-piperazine-1-carboxylate (617 mg, 1.43 mmol), 1,2,3,5,6,7-hexahydropyrrolizin-8-ylmethanol (608 mg, 4.30 mmol) and DIEA (556 mg, 4.30 mmol) in dioxane (6 mL) was degassed and purged with N₂ 3 times and then the mixture was stirred at 90° C. for 16 h under N₂ atmosphere. The mixture was concentrated to dryness then partitioned between H₂O (60 mL) and ethyl acetate (60 mL). The organic phase was separated and the aqueous phase was extracted with ethyl acetate (60 mL×2). The combine organic layers were washed with brine (60 mL), dried over Na₂SO₄, filtered and concentrated under vacuum. The residue was purified by flash column chromatography on silica gel (MeOH in DCM from 0% to 6%) to afford tert-butyl (2S, 6R)-4-[7-chloro-8-fluoro-2-(1,2,3,5,6,7-hexahydropyrrolizin-8-ylmethoxy)pyrido[4,3-d]pyrimidin-4-yl]-2,6-dimethyl-piperazine-1-carboxylate (582 mg, 74% yield) as a yellow solid.

### Procedure for Preparation of Compound 6

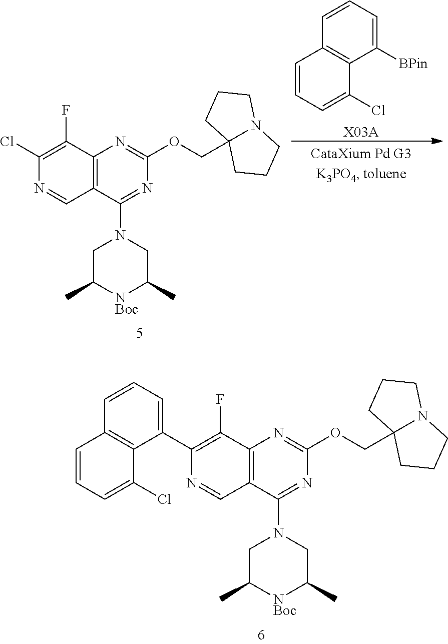

To a mixture of 2-(8-chloro-1-naphthyl)-4,4,5,5-tetramethyl-1,3,2-dioxaborolane (307 mg, 1.07 mmol) and tert-butyl (2R, 6S)-4-[7-chloro-8-fluoro-2-(1,2,3,5,6,7-hexahydropyrrolizin-8-ylmethoxy) pyrido[4,3-d]pyrimidin-4-yl]-2,6-dimethyl-piperazine-1-carboxylate (380 mg, 0.710 mmol) in dioxane (10 mL) and H₂O (5 mL) was added [2-(2-aminophenyl)phenyl]palladium(1+);bis(1-adamantyl)-butyl-phosphane; methanesulfonate (103 mg, 0.142 mmol) and K₃PO₄ (302 mg, 1.42 mmol) in one portion at 25° C. under N₂. The mixture was stirred at 100° C. for 6 h then filtered and concentrated in vacuo to give a residue which was purified by flash column chromatography on silica gel (MeOH in DCM from 0% to 20%) to afford tert-butyl (2R, 6S)-4-[7-(8-chloro-1-naphthyl)-8-fluoro-2-(1,2,3,5,6,7-hexahydropyrrolizin-8-ylmethoxy)pyrido[4,3-d]pyrimidin-4-yl]-2,6-dimethyl-piperazine-1-carboxylate (84 mg, 16% yield,) as a yellow solid.

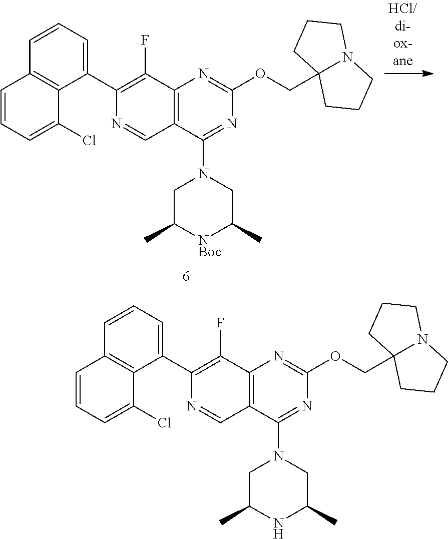 [SMILES: C[C@@H]1CN(c2nc(OCC34CCCN3CCC4)nc3c(F)c(-c4cccc5cccc(Cl)c45)ncc23)C[C@H](C)N1C(=O)OC(C)(C)C ; C[C@@H]1CN(c2nc(OCC34CCCN3CCC4)nc3c(F)c(-c4cccc5cccc(Cl)c45)ncc23)C[C@H](C)N1]

A solution of tert-butyl (2R, 6S)-4-[7-(8-chloro-1-naphthyl)-8-fluoro-2-(1,2,3,5,6,7-hexahydropyrrolizin-8-ylmethoxy)pyrido[4,3-d]pyrimidin-4-yl]-2,6-dimethyl-piperazine-1-carboxylate (84 mg, 0.127 mmol) in HCl/dioxane (4 M, 3 mL) was stirred at 25° C. for 0.5 h. The mixture was concentrated in vacuo to give a residue which was purified by prep-HPLC (column: C18-6 100*30 mm*5 um; mobile phase: [water (FA)-ACN]; B %: 12%-42%, 15 min) to afford 7-(8-chloronaphthalen-1-yl)-4-((3R,5S)-3,5-dimethylpiperazin-1-yl)-8-fluoro-2-((tetrahydro-1H-pyrrolizin-7a(5H)-yl)methoxy)pyrido[4,3-d]pyrimidine (18.2 mg, 24% yield) as a yellow solid. MS (ESI) m/z=561.5 [M+H]⁺. ¹H NMR (400 MHz, DMSO-d₆): δ=9.18-9.08 (m, 1H), 8.23-8.04 (m, 3H), 7.74-7.49 (m, 4H), 4.48-4.40 (m, 2H), 4.17 (m, 2H), 3.08-2.90 (m, 6H), 2.67 (m, 2H), 1.95 (m, 2H), 1.84 (m, 4H), 1.67-1.64 (m, 2H), 1.09-1.06 (m, 6H).

### Example 2

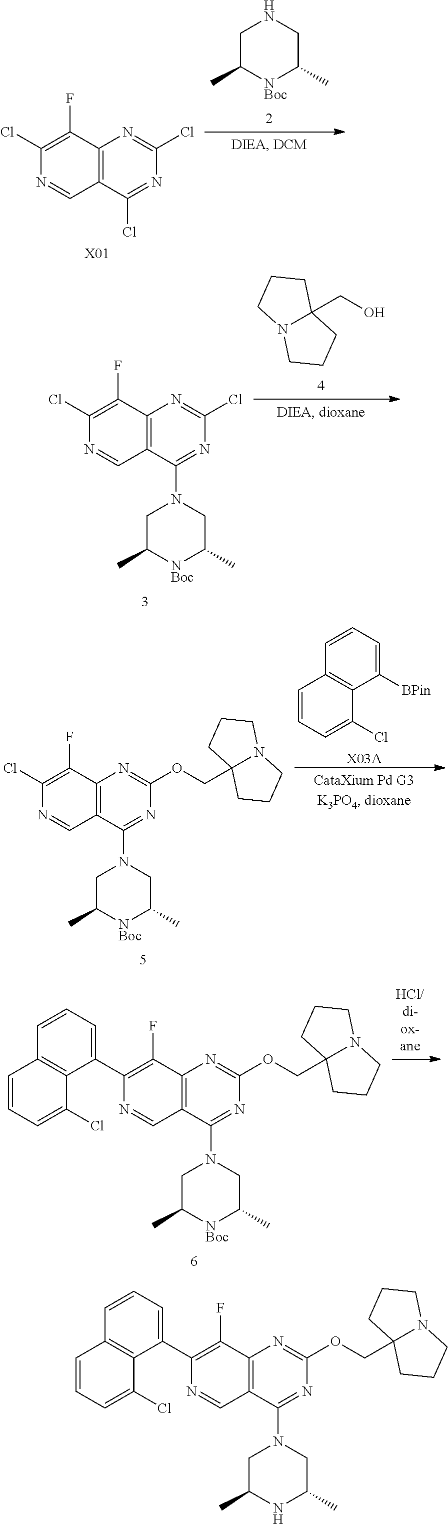

### Procedure for Preparation of Compound 3

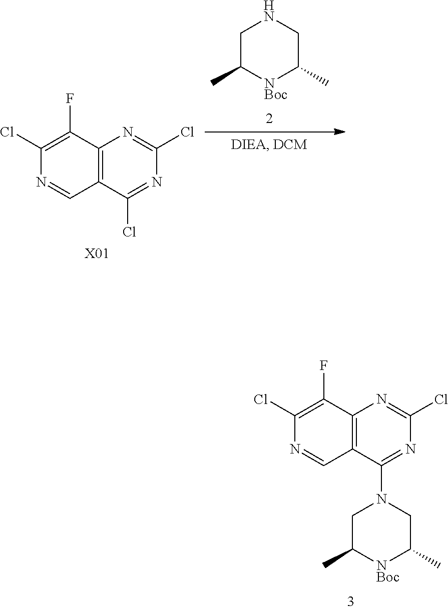

To a solution of 2,4,7-trichloro-8-fluoro-pyrido[4,3-d]pyrimidine (350 mg, 1.39 mmol) in DCM (20 mL) was added tert-butyl (2S, 6S)-2,6-dimethylpiperazine-1-carboxylate (267 mg, 1.25 mmol) and DIEA (1.79 g, 13.86 mmol) at −40° C. The mixture was stirred at −40° C. for 0.5 h then concentrated under reduced pressure. The residue was purified by flash column chromatography on silica gel (ethyl acetate in petroleum ether from 0% to 52%) to afford tert-butyl (2S, 6S)-4-(2,7-dichloro-8-fluoro-pyrido[4,3-d]pyrimidin-4-yl)-2,6-dimethyl-piperazine-1-carboxylate (270 mg, 0.565 mmol, 41% yield) as yellow solid.

### Procedure for Preparation of Compound 5

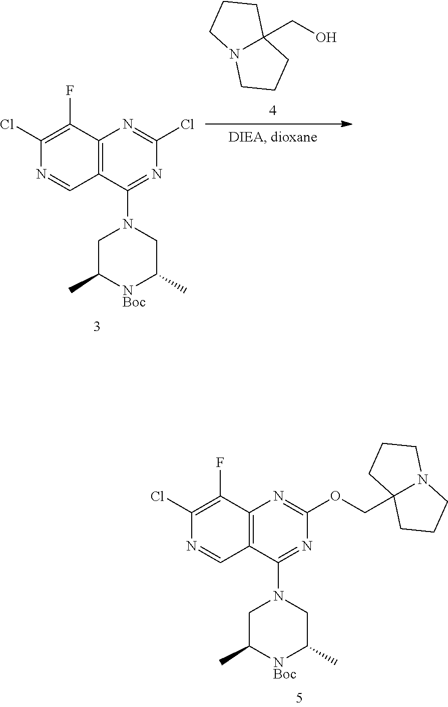 [SMILES: OCC12CCCN1CCC2 ; C[C@H]1CN(c2nc(Cl)nc3c(F)c(Cl)ncc23)C[C@H](C)N1C(=O)OC(C)(C)C ; C[C@H]1CN(c2nc(OCC34CCCN3CCC4)nc3c(F)c(Cl)ncc23)C[C@H](C)N1C(=O)OC(C)(C)C]

To a solution of tert-butyl (2S, 6S)-4-(2,7-dichloro-8-fluoro-pyrido[4,3-d]pyrimidin-4-yl)-2,6-dimethyl-piperazine-1-carboxylate (270 mg, 0.627 mmol) in dioxane (5 mL) was added 1,2,3,5,6,7-hexahydropyrrolizin-8-ylmethanol (266 mg, 1.88 mmol) and DIEA (243 mg, 1.88 mmol). The mixture was stirred at 90° C. for 4 h then concentrated in vacuo to give a residue. The residue was purified by flash column chromatography on silica gel (MeOH in DCM from 0% to 8%) to afford tert-butyl (2S, 6S)-4-[7-chloro-8-fluoro-2-(1,2,3,5,6,7-hexahydropyrrolizin-8-ylmethoxy)pyrido[4,3-d]pyrimidin-4-yl]-2,6-dimethyl-piperazine-1-carboxylate (290 mg, 0.488 mmol, 78% yield) as yellow solid.

### Procedure for Preparation of Compound 6

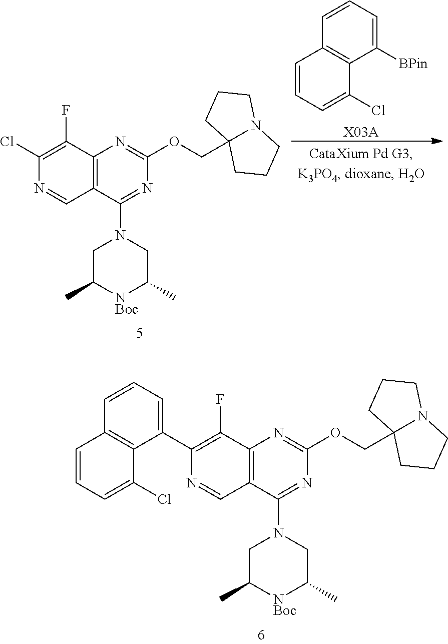

To a solution of tert-butyl (2S, 6S)-4-[7-chloro-8-fluoro-2-(1,2,3,5,6,7-hexahydropyrrolizin-8-ylmethoxy)pyrido[4,3-d]pyrimidin-4-yl]-2,6-dimethyl-piperazine-1-carboxylate (190 mg, 0.355 mmol) and 2-(8-chloro-1-naphthyl)-4,4,5,5-tetramethyl-1,3,2-dioxaborolane (154 mg, 0.533 mmol) in dioxane (5 mL) and H₂O (1 mL) was added [2-(2-aminophenyl)phenyl]palladium(1+);bis(1-adamantyl)-butyl-phosphane;methanesulfonate (52 mg, 0.071 mmol) and K₃PO₄ (151 mg, 0.710 mmol) under a N₂ atmosphere. The mixture was stirred at 100° C. for 4 h then quenched with H₂O (5 mL) and extracted with EtOAc (20 mL×3). Then combined organic layers, washed with sat. aq. NaCl (20 mL), dried over Na₂SO₄, filtered and concentrated. The residue was purified by prep-HPLC (column: Xtimate C 18 100*30 mm*10 um; mobile phase: [water (FA)-ACN];B %: 30%-60%, 10 min) to afford tert-butyl (2S, 6S)-4-[7-(8-chloro-1-naphthyl)-8-fluoro-2-(1,2,3,5,6,7-hexahydropyrrolizin-8-ylmethoxy)pyrido[4,3-d]pyrimidin-4-yl]-2,6-dimethyl-piperazine-1-carboxylate (25 mg, 0.036 mmol, 10% yield) as white solid.

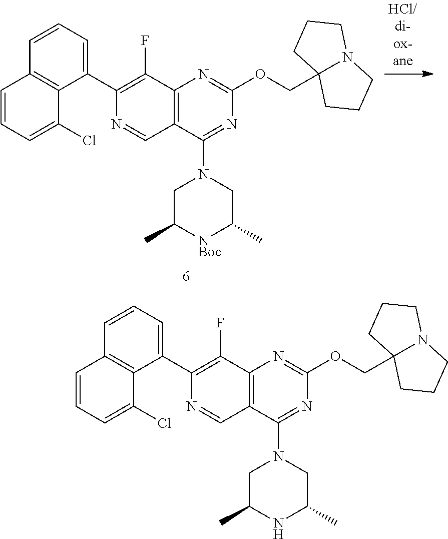 [SMILES: C[C@H]1CN(c2nc(OCC34CCCN3CCC4)nc3c(F)c(-c4cccc5cccc(Cl)c45)ncc23)C[C@H](C)N1C(=O)OC(C)(C)C ; C[C@H]1CN(c2nc(OCC34CCCN3CCC4)nc3c(F)c(-c4cccc5cccc(Cl)c45)ncc23)C[C@H](C)N1]

A solution of tert-butyl (2S, 6S)-4-[7-(8-chloro-1-naphthyl)-8-fluoro-2-(1,2,3,5,6,7-hexahydropyrrolizin-8-ylmethoxy)pyrido[4,3-d]pyrimidin-4-yl]-2,6-dimethyl-piperazine-1-carboxylate (25 mg, 0.038 mmol) in HCl/dioxane (4 M, 2 mL) was stirred at 20° C. for 1 h. The mixture was adjusted to pH ˜8 with sat. NaHCO₃. The mixture was extracted with EtOAc (10 mL×3) and the organic layers were combined and washed with brine (10 mL×2), dried over Na₂SO₄, filtered and concentrated under reduced pressure. The residue was purified by prep-HPLC (column: Xtimate C 18 100*30 mm*10 um; mobile phase: [water (FA)-ACN]; B %: 10%-40%, 10 min) to afford 7-(8-chloronaphthalen-1-yl)-4-((3S,5S)-3,5-dimethylpiperazin-1-yl)-8-fluoro-2-((tetrahydro-1H-pyrrolizin-7a(5H)-yl)methoxy)pyrido[4,3-d]pyrimidine (15.3 mg, 71% yield) as yellow solid. MS (ESI) m/z=552.4 [M+H]⁺. ¹H NMR: ¹H NMR (400 MHz, DMSO-d₆): δ ppm=1.13-1.08 (m, 6H), 1.53-2.01 (m, 8H), 2.58-2.64 (m, 2H), 2.96-3.04 (m, 2H), 3.37-3.34 (m, 2H), 3.67-3.72 (m, 2H), 3.95-4.01 (m, 2H), 4.10-4.18 (m, 2H), 7.48-7.77 (m, 4H), 8.03-8.25 (m, 3H), 9.03-9.28 (m, 1H).

### Example 3

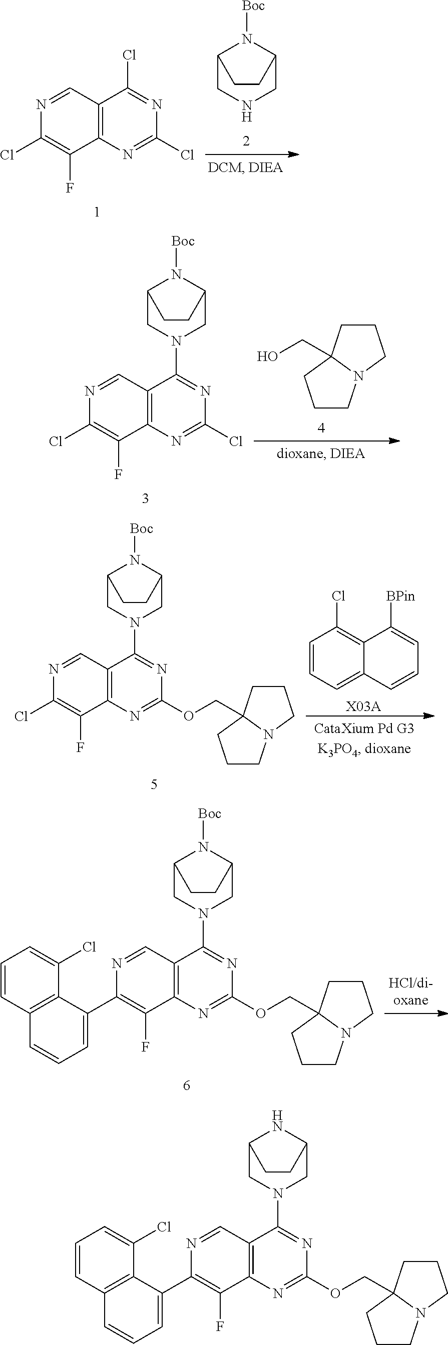

### Procedure for Preparation of Compound 3

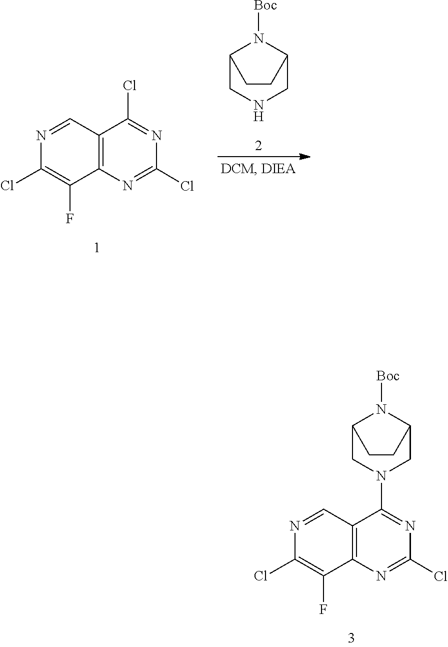 [SMILES: Fc1c(Cl)ncc2c(Cl)nc(Cl)nc12 ; CC(C)(C)OC(=O)N1C2CCC1CNC2 ; CC(C)(C)OC(=O)N1C2CCC1CN(c1nc(Cl)nc3c(F)c(Cl)ncc13)C2]

To a solution of 2,4,7-trichloro-8-fluoro-pyrido[4,3-d]pyrimidine (1.5 g, 5.94 mmol) in DCM (35 mL) was added DIEA (7.68 g, 59.42 mmol) and tert-butyl 3,8-diazabicyclo[3.2.1]octane-8-carboxylate (1.14 g, 5.35 mmol) at −40° C. The mixture was stirred at −40° C. for 0.5 h then concentrated. The residue was partitioned between H₂O (60 mL) and DCM (60 mL). The organic phase was separated and the aqueous phase was extracted with DCM (60 mL×2). The combine organic layers were dried over Na₂SO₄, filtered and concentrated under vacuum. The residue was purified by flash column chromatography on silica gel (ethyl acetate in petroleum ether from 0% to 28%) to afford tert-butyl 3-(2,7-dichloro-8-fluoro-pyrido[4,3-d]pyrimidin-4-yl)-3,8-diazabicyclo[3.2.1]octane-8-carboxylate (1.7 g, 3.30 mmol, 56% yield) as a yellow solid.

### Procedure for Preparation of Compound 5

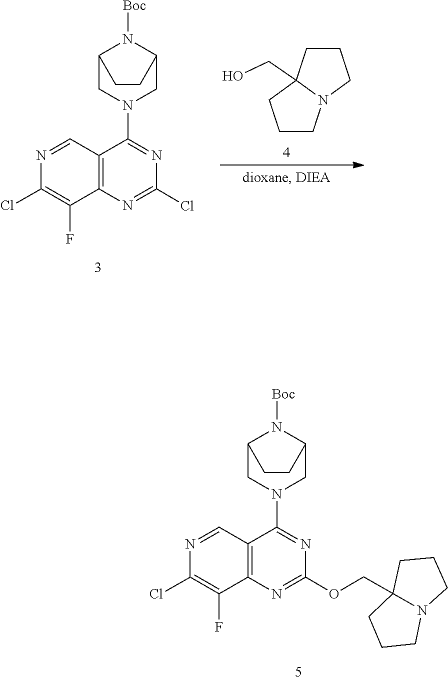 [SMILES: CC(C)(C)OC(=O)N1C2CCC1CN(c1nc(Cl)nc3c(F)c(Cl)ncc13)C2 ; OCC12CCCN1CCC2 ; CC(C)(C)OC(=O)N1C2CCC1CN(c1nc(OCC34CCCN3CCC4)nc3c(F)c(Cl)ncc13)C2]

To a solution of tert-butyl 3-(2,7-dichloro-8-fluoro-pyrido[4,3-d]pyrimidin-4-yl)-3,8-diazabicyclo[3.2.1]octane-8-carboxylate (500 mg, 1.17 mmol) and 1,2,3,5,6,7-hexahydropyrrolizin-8-ylmethanol (495 mg, 3.50 mmol) in dioxane (12 mL) was added DIEA (453 mg, 3.50 mmol). The mixture was stirred at 90° C. for 16 hr. The mixture was concentrated to dryness. The residue was partitioned between H₂O (30 mL) and ethyl acetate (30 mL). The organic phase was separated, and the aqueous phase was extracted with ethyl acetate (30 mL×2). The combine organic layers were with brine (90 mL), dried over Na₂SO₄, filtered and concentrated under vacuum. The residue was purified by flash column chromatography on silica gel (methanol in dichloromethane from 0% to 4%) to afford tert-butyl 3-[7-chloro-8-fluoro-2-(1,2,3,5,6,7-hexahydropyrrolizin-8-ylmethoxy)pyrido[4,3-d]pyrimidin-4-yl]-3,8-diazabicyclo[3.2.1]octane-8-carboxylate (407 mg, 64% yield) as a yellow solid.

### Procedure for Preparation of Compound 6

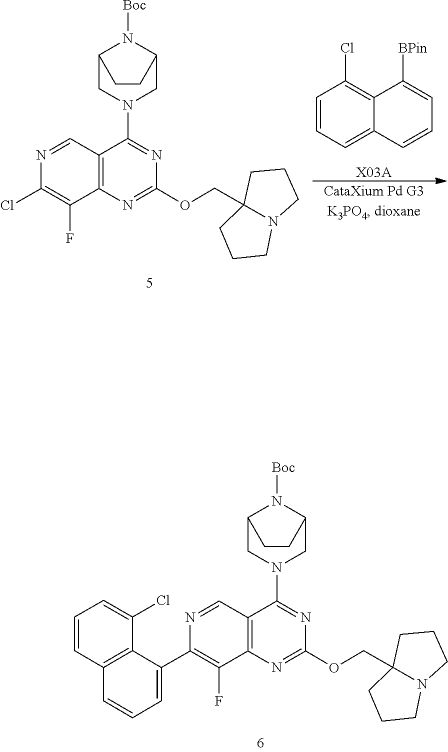

A mixture of tert-butyl 3-[7-chloro-8-fluoro-2-(1,2,3,5,6,7-hexahydropyrrolizin-8-ylmethoxy)pyrido[4,3-d]pyrimidin-4-yl]-3,8-diazabicyclo[3.2.1]octane-8-carboxylate (307 mg, 0.575 mmol), 2-(8-chloro-1-naphthyl)-4,4,5,5-tetramethyl-1,3,2-dioxaborolane (249 mg, 0.863 mmol), K₃PO₄ (245 mg, 1.15 mmol), [2-(2-aminophenyl)phenyl]palladium(1+);bis(1-adamantyl)-butyl-phosphane;methanesulfonate (84 mg, 0.115 mmol) in dioxane (8 mL) and H₂O (4 mL) was degassed and purged with N₂ 3 times then the mixture was stirred at 100° C. for 6 h under N₂ atmosphere. The mixture was concentrated under reduced pressure to give a residue which was purified by flash column chromatography on silica gel (methanol in dichloromethane from 0% to 12%) to afford tert-butyl 3-[7-(8-chloro-1-naphthyl)-8-fluoro-2-(1,2,3,5,6,7-hexahydropyrrolizin-8-ylmethoxy)pyrido[4,3-d]pyrimidin-4-yl]-3,8-diazabicyclo[3.2.1]octane-8-carboxylate (140 mg, 24% yield) as a yellow solid.

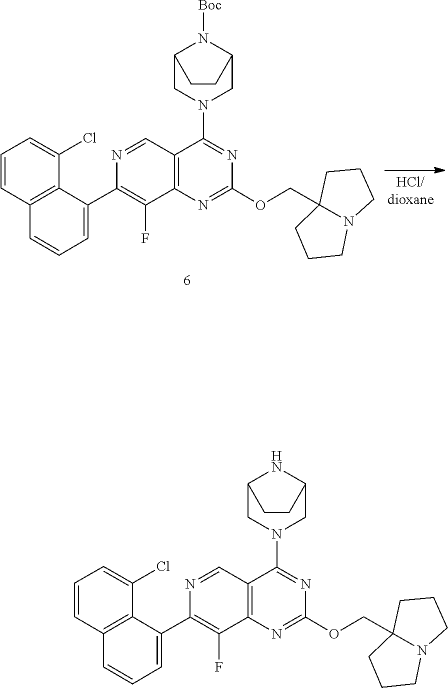 [SMILES: CC(C)(C)OC(=O)N1C2CCC1CN(c1nc(OCC34CCCN3CCC4)nc3c(F)c(-c4cccc5cccc(Cl)c45)ncc13)C2 ; Fc1c(-c2cccc3cccc(Cl)c23)ncc2c(N3CC4CCC(C3)N4)nc(OCC34CCCN3CCC4)nc12]

A solution of tert-butyl 3-[7-(8-chloro-1-naphthyl)-8-fluoro-2-(1,2,3,5,6,7-hexahydropyrrolizin-8-ylmethoxy)pyrido[4,3-d]pyrimidin-4-yl]-3,8-diazabicyclo[3.2.1]octane-8-carboxylate (140 mg, 0.211 Mmol) in HCl/dioxane (4 M, 2 mL) was stirred at 25° C. for 0.5 h. The mixture was concentrated under reduced pressure to give a residue which was purified by prep-HPLC (column: C18-6 100*30 mm*5 um; mobile phase: [water (FA)-ACN]; B %: 0%-35%, 15 min); FlowRate (25 mL/min)) to afford 4-((1R,5S)-3,8-diazabicyclo[3.2.1]octan-3-yl)-7-(8-chloronaphthalen-1-yl)-8-fluoro-2-((tetrahydro-1H-pyrrolizin-7a(5H)-yl)methoxy)pyrido[4,3-d]pyrimidine (51.6 mg, 43% yield) as a yellow solid. MS (ESI) m/z=559.5 [M+H]⁺. ¹H NMR (400 MHz, DMSO-d₆): δ=9.19-9.09 (d, J 39.6 HZ, 1H), 8.22-8.05 (m, 4H), 7.74-7.56 (m, 3H), 4.51-4.44 (t, J 14.0 HZ, 2H), 4.17-4.16 (d, J 6.0 HZ, 2H), 3.68-3.66 (m, 4H), 3.08-3.03 (m, 2H), 2.71-2.65 (m, 2H), 1.98-1.63 (m, 10H).

### Example 4

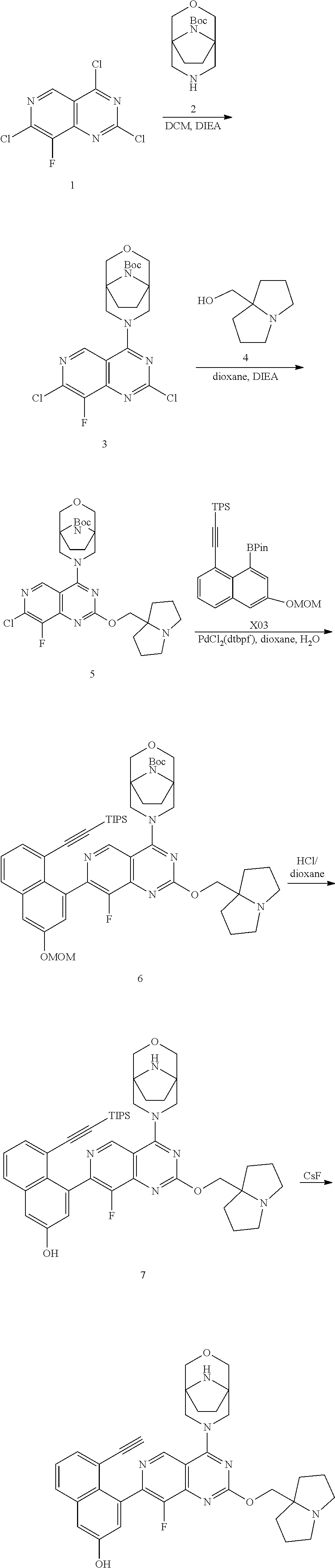

### Procedure for Preparation of Compound 3

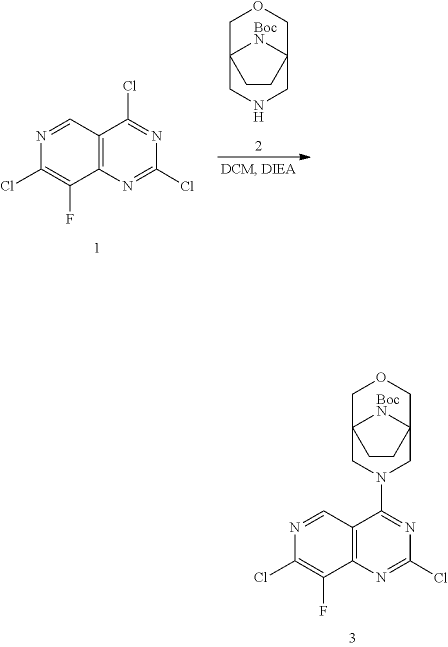 [SMILES: Fc1c(Cl)ncc2c(Cl)nc(Cl)nc12 ; CC(C)(C)OC(=O)N1C23CCC1(CNC2)COC3 ; CC(C)(C)OC(=O)N1C23CCC1(COC2)CN(c1nc(Cl)nc2c(F)c(Cl)ncc12)C3]

To a solution of tert-butyl 3-oxa-7,9-diazabicyclo[3.3.1]nonane-9-carboxylate (434 mg, 1.90 mmol), 2,4,7-trichloro-8-fluoro-pyrido[4,3-d]pyrimidine (480 mg, 1.90 mmol) in DCM (5 mL) was added DIEA (2.46 g, 19.01 mmol). The mixture was stirred at −40° C. for 0.5 h. The mixture was concentrated in vacuo to give a residue. To the mixture was added sat. aq. NH₄Cl (10 mL) and dichloromethane (10 mL) then it was extracted with dichloromethane (10 mL×3). The combined organic layers were evaporated to dryness and the residue was purified by flash column chromatography on silica gel (MeOH in DCM from 0% to 10%) to afford tert-butyl 7-(2,7-dichloro-8-fluoro-pyrido[4,3-d]pyrimidin-4-yl)-3-oxa-7,9-diazabicyclo[3.3.1]nonane-9-carboxylate (630 mg, crude) as a yellow solid.

### Procedure for Preparation of Compound 5

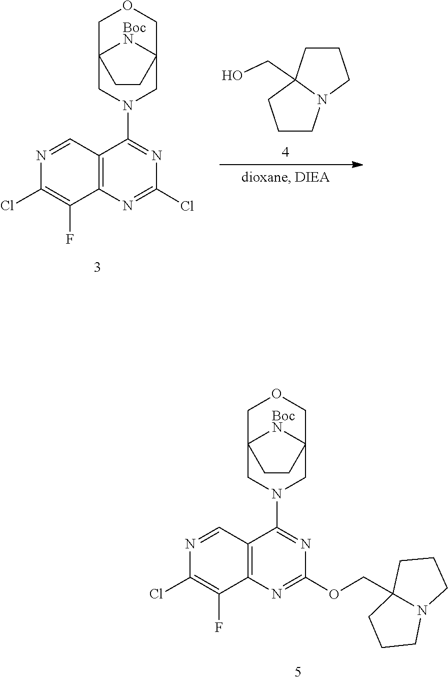 [SMILES: CC(C)(C)OC(=O)N1C23CCC1(COC2)CN(c1nc(Cl)nc2c(F)c(Cl)ncc12)C3 ; OCC12CCCN1CCC2 ; CC(C)(C)OC(=O)N1C23CCC1(COC2)CN(c1nc(OCC24CCCN2CCC4)nc2c(F)c(Cl)ncc12)C3]

To a solution of 1,2,3,5,6,7-hexahydropyrrolizin-8-ylmethanol (572 mg, 4.05 mmol), tert-butyl 7-(2,7-dichloro-8-fluoro-pyrido[4,3-d]pyrimidin-4-yl)-3-oxa-7,9-diazabicyclo[3.3.1]nonane-9-carboxylate (600 mg) in dioxane (5 mL) was added DIEA (524 mg, 4.05 mmol). The mixture was stirred at 90° C. for 16 h. The mixture was then concentrated and the residue was purified by flash column chromatography on silica gel (MeOH in DCM from 0% to 5%) to afford tert-butyl 7-[7-chloro-8-fluoro-2-(1,2,3,5,6,7-hexahydropyrrolizin-8-ylmethoxy)pyrido[4,3-d]pyrimidin-4-yl]-3-oxa-7,9-diazabicyclo[3.3.1]nonane-9-carboxylate (330 mg, 43% yield) as a yellow solid.

### Procedure for Preparation of Compound 6

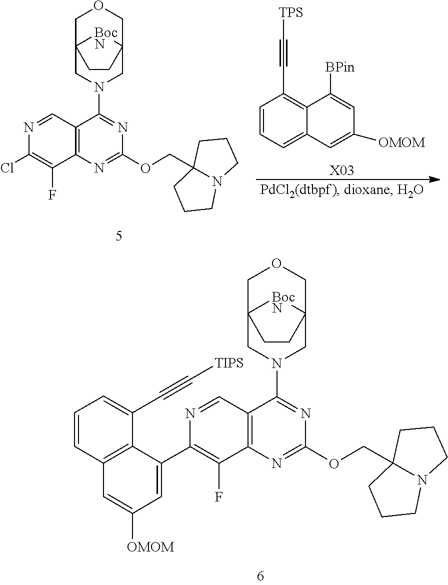

To a solution of tert-butyl 7-[7-chloro-8-fluoro-2-(1,2,3,5,6,7-hexahydropyrrolizin-8-ylmethoxy)pyrido[4,3-d]pyrimidin-4-yl]-3-oxa-7,9-diazabicyclo[3.3.1]nonane-9-carboxylate (210 mg, 0.382 mmol) triisopropyl-[2-[6-(methoxymethoxy)-8-(4,4,5,5-tetramethyl-1,3,2-dioxaborolan-2-yl)-1-naphthyl]ethynyl]silane (208 mg, 0.420 mmol) in dioxane (5 mL) and H₂O (1 mL) was added ditert-butyl(cyclopentyl)phosphane;dichloropalladium;iron (50 mg, 0.076 mmol) and Cs₂CO₃ (349 mg, 1.07 mmol). The mixture was stirred at 100° C. for 16 h under N₂ then concentrated in vacuo to give a residue. To the mixture was added sat. aq. NH₄Cl (10 mL) and dichloromethane (10 mL) then it was extracted with dichloromethane (10 mL×3). The combined organic layers were evaporated to dryness. The residue was purified by flash column chromatography on silica gel (MeOH in DCM from 0% to 10%) to afford tert-butyl 7-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-3-oxa-7,9-diazabicyclo[3.3.1]nonane-9-carboxylate (70 mg, crude) as a brown solid. MS (ESI) m/z=304.2 [M+H]⁺.

### Procedure for Preparation of Compound 7

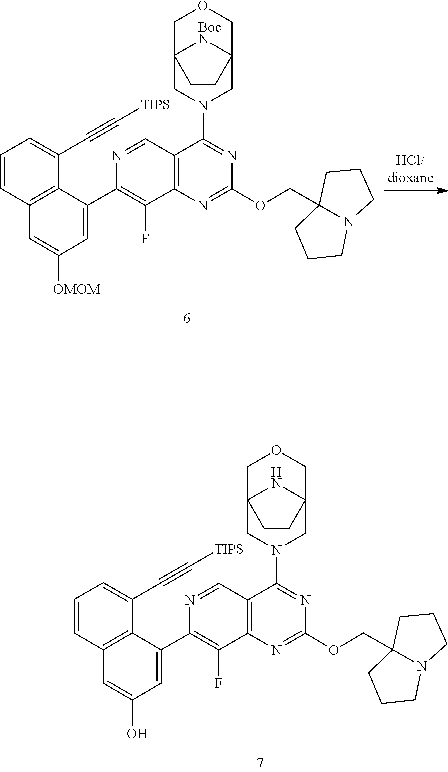

To a mixture of tert-butyl 7-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-3-oxa-7,9-diazabicyclo[3.3.1]nonane-9-carboxylate (70 mg, 0.079 mmol) in dioxane (1 mL) was added HCl (12 M, 0.33 mL) in one portion at 0° C. The mixture was stirred at 25° C. for 1 h then it was adjusted to pH ˜10 by addition of saturated aqueous NaHCO₃ (10 mL). The resulting mixture was extracted with DCM (20 mL×3). The combined organic phases were washed with brine (30 mL), dried with anhydrous Na₂SO₄, filtered and concentrated under vacuum to afford 4-[8-fluoro-2-(1,2,3,5,6,7-hexahydropyrrolizin-8-ylmethoxy)-4-(3-oxa-7,9-diazabicyclo[3.3.1]nonan-7-yl)pyrido[4,3-d]pyrimidin-7-yl]-5-(2-triisopropylsilylethynyl)naphthalen-2-ol (57 mg, crude) as a black brown solid.

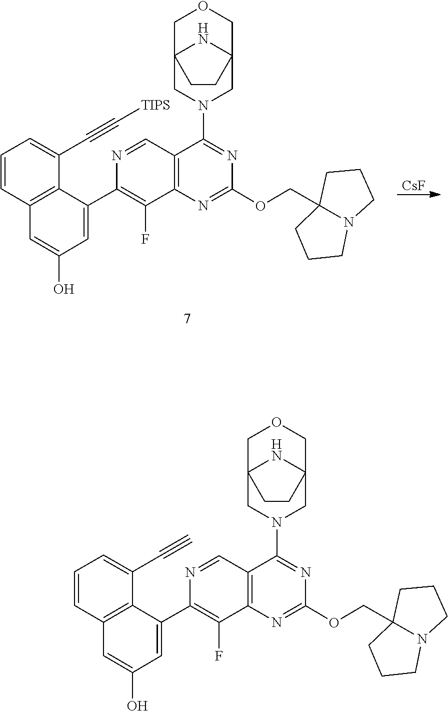

To a mixture of 4-[8-fluoro-2-(1,2,3,5,6,7-hexahydropyrrolizin-8-ylmethoxy)-4-(3-oxa-7,9-diazabicyclo[3.3.1]nonan-7-yl)pyrido[4,3-d]pyrimidin-7-yl]-5-(2-triisopropylsilylethynyl)naphthalen-2-ol (57 mg, 0.077 mmol) in DMF (2 mL) was added CsF (117 mg, 0.772 mmol) in one portion at 25° C. The mixture was stirred at 25° C. for 12 h then filtered and the filtrate was purified by prep-HPLC (column: C18-6 100*30 mm*5 um; mobile phase: [water (FA)-ACN]; B %: 0%-35%, 15 min); FlowRate (25 mL/min)) to afford 4-(4-(3-oxa-7,9-diazabicyclo[3.3.1]nonan-7-yl)-8-fluoro-2-((tetrahydro-1H-pyrrolizin-7a(5H)-yl)methoxy)pyrido[4,3-d]pyrimidin-7-yl)-5-ethynylnaphthalen-2-ol (13.6 mg, 30% yield) as a yellow solid. MS (ESI) m/z=581.5 [M+H]⁺. ¹H NMR (400 MHz, DMSO-d₆): δ=9.09 (s, 1H), 8.22 (s, 1H), 7.89-7.87 (d, J 8.0 HZ, 1H), 7.47-7.39 (m, 1H), 7.34-7.33 (m, 1H), 7.12-7.11 (d, J 2.4 HZ, 1H), 4.79-7.71 (m, 2H), 4.15-4.09 (m, 2H), 3.88-3.72 (m, 6H), 3.53 (s, 2H), 3.12-2.95 (m, 4H), 2.76-2.63 (m, 2H), 2.00-1.75 (m, 5H), 1.67-1.45 (m, 1H), 1.22-0.84 (m, 2H).

### Example 5

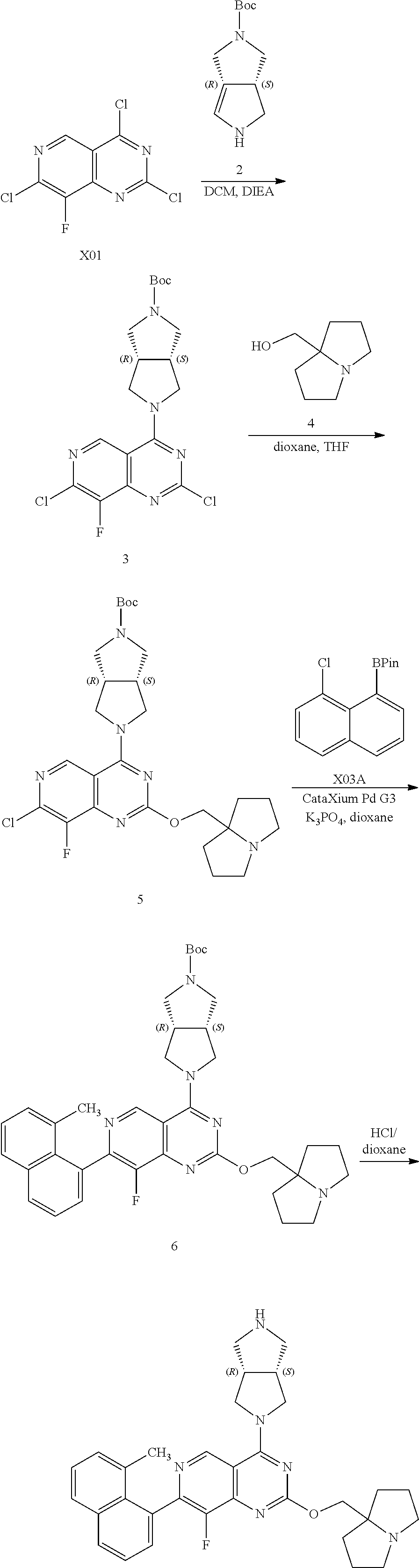

### Procedure for Preparation of Compound 3

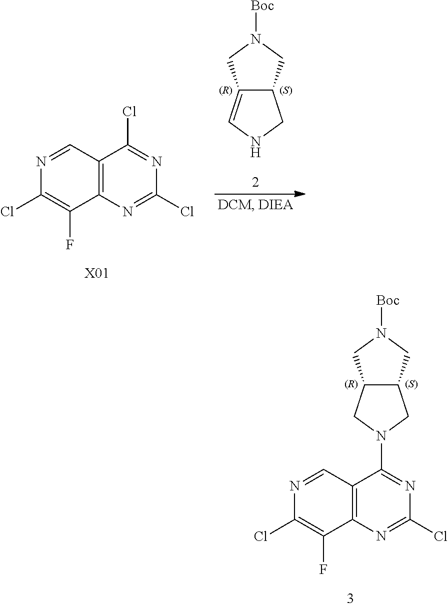

To a solution of 2,4,7-trichloro-8-fluoro-pyrido[4,3-d]pyrimidine (621 mg, 2.21 mmol) in DCM (20 mL) was added DIEA (2.86 g, 22.14 mmol) and tert-butyl (3aS, 6aR)-2,3,3a,4,6,6a-hexahydro-1H-pyrrolo[3,4-c]pyrrole-5-carboxylate (470 mg, 2.21 mmol) at −40° C. The mixture was stirred at −40° C. for 0.5 h. The mixture was concentrated in vacuo. The residue was purified by flash silica gel chromatography (ISCO@; 12 g SepaFlash® Silica Flash Column, Eluent of 0-39% Ethyl acetate/Petroleum ether gradient @30 mL/min) to afford tert-butyl (3aS, 6aR)-2-(2,7-dichloro-8-fluoro-pyrido[4,3-d]pyrimidin-4-yl)-1,3,3a,4,6,6a-hexahydropyrrolo[3,4-c]pyrrole-5-carboxylate (800 mg, 84% yield) as a yellow solid. ¹H NMR (400 MHz, CDCl₃): δ=9.08, 9.02 (s, 1H), 4.34-4.18 (m, 2H), 3.96-3.90 (m, 2H), 3.76-3.66 (m, 2H), 3.46−3.32 (m, 2H), 3.22-2.98 (m, 2H), 1.50-1.46 (m, 9H).

### Procedure for Preparation of Compound 5

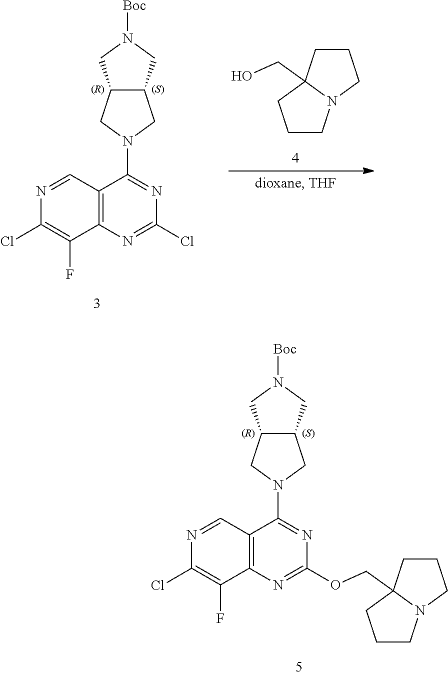

To a solution of tert-butyl (3aS, 6aR)-2-(2,7-dichloro-8-fluoro-pyrido[4,3-d]pyrimidin-4-yl)-1,3,3a,4,6,6a-hexahydropyrrolo[3,4-c]pyrrole-5-carboxylate (800 mg, 1.87 mmol) in THE (2 mL) was added DIEA (724 mg, 5.60 mmol) and 1,2,3,5,6,7-hexahydropyrrolizin-8-ylmethanol (791 mg, 5.60 mmol). The mixture was stirred at 65° C. for 16 h. The mixture was concentrated in vacuo. The residue was purified by flash silica gel chromatography (ISCO®; 12 g SepaFlash® Silica Flash Column, Eluent of 0˜10% MeOH/DCM@30 mL/min) to afford tert-butyl (3aS, 6aR)-2-[7-chloro-8-fluoro-2-(1,2,3,5,6,7-hexahydropyrrolizin-8-ylmethoxy)pyrido[4,3-d]pyrimidin-4-yl]-1,3,3a,4,6,6a-hexahydropyrrolo[3,4-c]pyrrole-5-carboxylate (950 mg, 95% yield) as a yellow solid. 1H NMR (400 MHz, CDCl₃): δ=8.92 (s, 1H), 3.84-3.78 (m, 2H), 3.70 (s, 2H), 3.40-3.32 (m, 2H), 2.96-2.90 (m, 2H), 2.86-2.78 (m, 2H), 2.26-2.00 (m, 12H), 1.84-1.80 (m, 2H), 1.46 (s, 9H).

### Procedure for Preparation of Compound 6

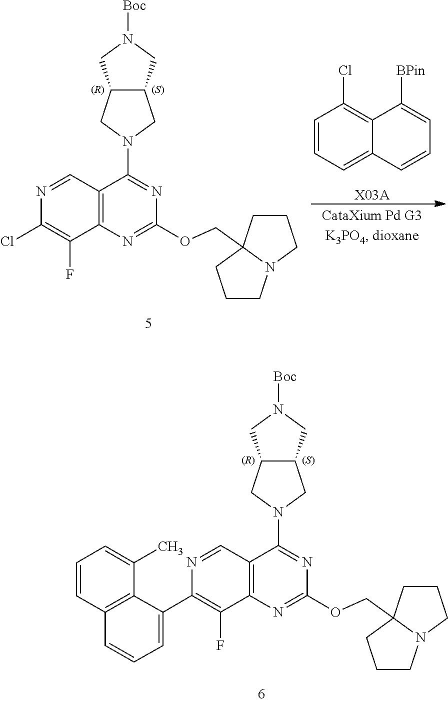

To a solution of tert-butyl (3aS, 6aR)-2-[7-chloro-8-fluoro-2-(1,2,3,5,6,7-hexahydropyrrolizin-8-ylmethoxy)pyrido[4,3-d]pyrimidin-4-yl]-1,3,3a,4,6,6a-hexahydropyrrolo[3,4-c]pyrrole-5-carboxylate (232 mg, 0.435 mmol) in dioxane (3 mL) and H₂O (1.5 mL) was added K₃PO₄ (185 mg, 0.87 mmol), [2-(2-aminophenyl)phenyl]palladium(1+);bis(1-adamantyl)-butyl-phosphane;methanesulfonate (63 mg, 0.087 mmol) and 2-(8-chloro-1-naphthyl)-4,4,5,5-tetramethyl-1,3,2-dioxaborolane (188 mg, 0.65 mmol). The mixture was stirred at 100° C. for 3 h under N₂ then filtered and the filtrate was concentrated in vacuo. The residue was purified by prep-HPLC (column: C18-6 100*30 mm*5 um;mobile phase: [water(FA)-ACN]; B %: 10%-60%, 15 min); FlowRate (25 mL/min)) to afford tert-butyl (3aS, 6aR)-2-[7-(8-chloro-1-naphthyl)-8-fluoro-2-(1,2,3,5,6,7-hexahydropyrrolizin-8-ylmethoxy)pyrido[4,3-d]pyrimidin-4-yl]-1,3,3a,4,6,6a-hexahydropyrrolo[3,4-c]pyrrole-5-carboxylate (50 mg, 16% yield) as a white solid. ¹H NMR (400 MHz, CDCl₃): δ=9.40, 9.10 (s, 1H), 8.02-7.92 (m, 1H), 7.90-7.84 (m, 1H), 7.64-7.38 (m, 4H), 4.86-4.62 (m, 2H), 4.40-4.24 (m, 2H), 4.04-3.86 (m, 3H), 3.82-3.64 (m, 2H), 3.52-3.30 (m, 2H), 3.24-2.88 (m, 4H), 2.46-2.34 (m, 2H), 2.28-2.20 (m, 2H), 2.14-2.06 (m, 2H), 2.04-1.94 (m, 4H), 1.48 (s, 9H).

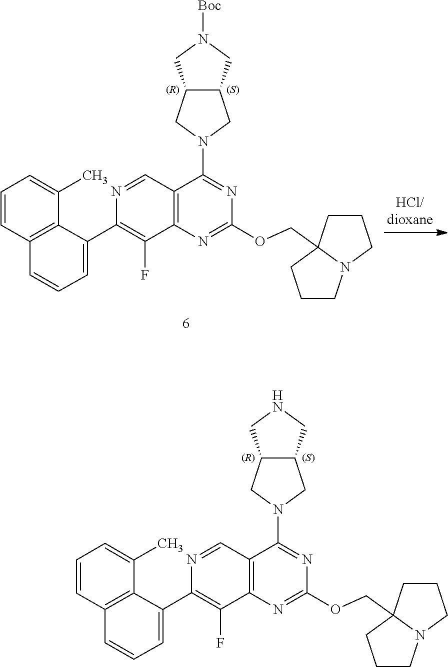

A solution of tert-butyl (3aS, 6aR)-2-[7-(8-chloro-1-naphthyl)-8-fluoro-2-(1,2,3,5,6,7-hexahydropyrrolizin-8-ylmethoxy)pyrido[4,3-d]pyrimidin-4-yl]-1,3,3a,4,6,6a-hexahydropyrrolo[3,4-c]pyrrole-5-carboxylate (50 mg, 0.075 mmol) in HCl/dioxane (4 M, 4 mL) was stirred at 25° C. for 0.5 h. The mixture was concentrated in vacuo. The residue was purified by prep-HPLC (column: C18-6 100*30 mm*5 um; mobile phase: [water (FA)-ACN];B %: 3%-33%, 15 min); FlowRate (25 mL/min)) to afford 4-[(3aR, 6aS)-2,3,3a,4,6,6a-hexahydro-1H-pyrrolo[3,4-c]pyrrol-5-yl]-7-(8-chloro-1-naphthyl)-8-fluoro-2-(1,2,3,5,6,7-hexahydropyrrolizin-8-ylmethoxy)pyrido[4,3-d]pyrimidine (15 mg, 34% yield) as a white solid. MS (ESI) m/z=559.2 [M+H]⁺. ¹H NMR (400 MHz, CDCl₃): δ=9.38-9.10 (m, 1H), 8.47-8.42 (m, 1H), 8.00-7.40 (m, 6H), 4.76-4.58 (m, 2H), 4.44-4.30 (m, 1H), 4.24-3.84 (m, 4H), 3.80-3.74 (m, 2H), 3.68-3.44 (m, 2H), 3.34-3.16 (m, 4H), 2.68-1.92 (m, 10H).

### Example 6

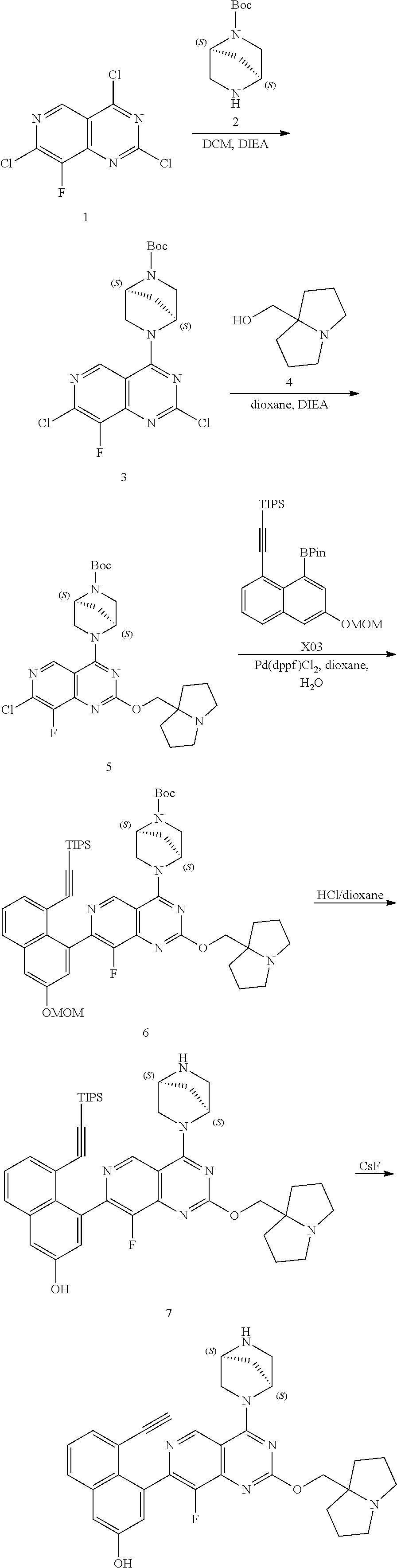

### Procedure for Preparation of Compound 3

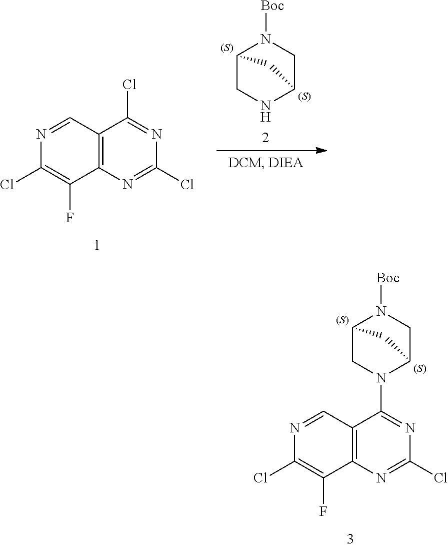

To a solution of 2,4,7-trichloro-8-fluoro-pyrido[4,3-d]pyrimidine (600 mg, 2.38 mmol) in DCM (20 mL) was added DIEA (3.07 g, 23.77 mmol) and tert-butyl (1S, 4S)-2,5-diazabicyclo[2.2.1]heptane-2-carboxylate (471 mg, 2.38 mmol). The mixture was stirred at −40° C. for 0.5 h. The mixture was concentrated in vacuo. The residue was purified by flash silica gel chromatography (ISCO®; 12 g SepaFlash® Silica Flash Column, Eluent of 0˜8% Dichloromethane/Methanol @60 mL/min) to afford tert-butyl (1S, 4S)-5-(2,7-dichloro-8-fluoro-pyrido[4,3-d]pyrimidin-4-yl)-2,5-diazabicyclo[2.2.1]heptane-2-carboxylate (1 g, 94% yield) as a yellow solid.

### Procedure for Preparation of Compound 5

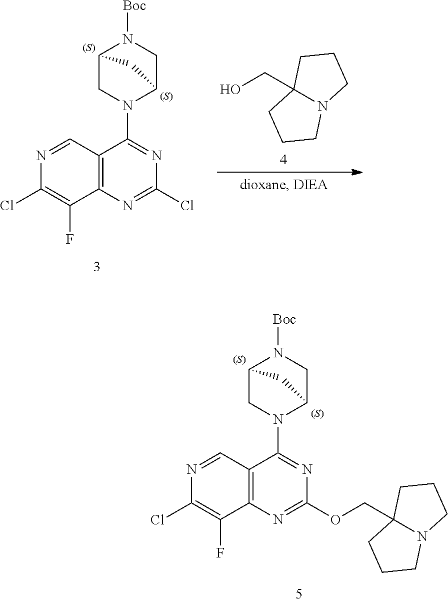

To a solution of tert-butyl (1S, 4S)-5-(2,7-dichloro-8-fluoro-pyrido[4,3-d]pyrimidin-4-yl)-2,5-diazabicyclo[2.2.1]heptane-2-carboxylate (1 g, 2.41 mmol) in dioxane (30 mL) was added DIEA (936 mg, 7.24 mmol) and 1,2,3,5,6,7-hexahydropyrrolizin-8-ylmethanol (1.02 g, 7.24 mmol). The mixture was stirred at 90° C. for 16 h then concentrated under reduced pressure. The residue was purified by flash silica gel chromatography (ISCO®; 12 g SepaFlash® Silica Flash Column, Eluent of 0˜3% Methanol/Dichloromethane @60 mL/min) to afford tert-butyl (1S, 4S)-5-[7-chloro-8-fluoro-2-(1,2,3,5,6,7-hexahydropyrrolizin-8-ylmethoxy)pyrido[4,3-d]pyrimidin-4-yl]-2,5-diazabicyclo[2.2.1]heptane-2-carboxylate (1.2 g, 83% yield) as a brown oil.

### Procedure for Preparation of Compound 6

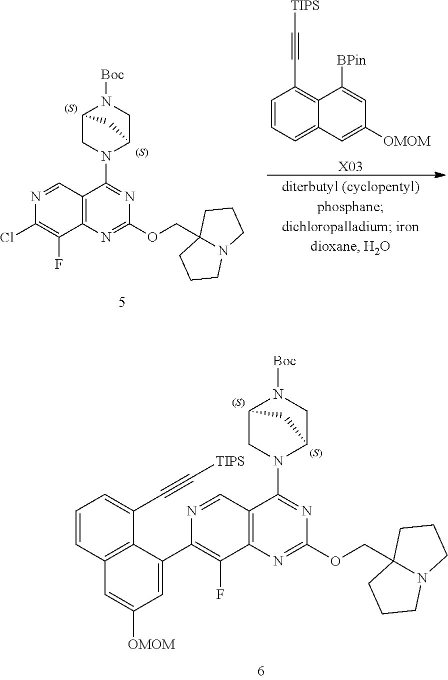

To a solution of tert-butyl (1S, 4S)-5-[7-chloro-8-fluoro-2-(1,2,3,5,6,7-hexahydropyrrolizin-8-ylmethoxy) pyrido[4,3-d]pyrimidin-4-yl]-2,5-diazabicyclo[2.2.1]heptane-2-carboxylate (500 mg, 0.96 mmol) in dioxane (20 mL) and H₂O (4 mL) was added Cs₂CO₃ (879 mg, 2.70 mmol), triisopropyl-[2-[6-(methoxymethoxy)-8-(4,4,5,5-tetramethyl-1,3,2-dioxaborolan-2-yl)-1-naphthyl]ethynyl]silane (548 mg, 1.11 mmol) and ditertbutyl (cyclopentyl) phosphane; dichloropalladium;iron (126 mg, 0.19 mmol). The mixture was stirred at 100° C. for 16 h. The mixture was concentrated under reduced pressure. The residue was purified by flash silica gel chromatography (ISCO®; 12 g SepaFlash® Silica Flash Column, Eluent of 0˜8% Dichloromethane/Methanol @50 mL/min) to afford tert-butyl(1S, 4S)-5-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-2,5-diazabicyclo[2.2.1]heptane-2-carboxylate (239 mg, 26% yield) as a brown solid.

### Procedure for Preparation of Compound 7

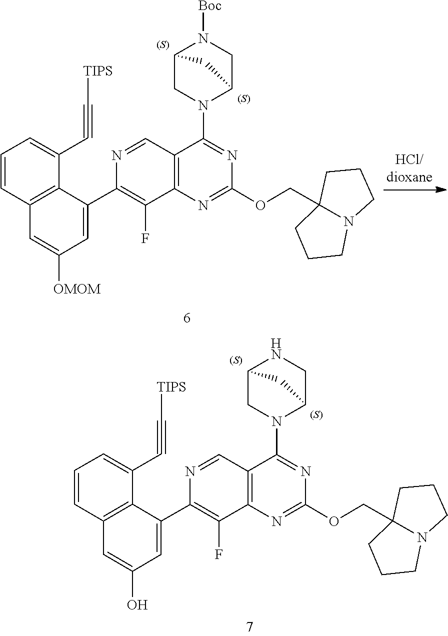

To a mixture of tert-butyl (1S, 4S)-5-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-2,5-diazabicyclo[2.2.1]heptane-2-carboxylate (250 mg, 0.29 mmol) in dioxane (6 mL) was added HCl (12 M, 2.0 mL) in one portion at 0° C. The mixture was stirred at 20° C. for 1 h then adjusted to pH ˜8 by addition of saturated aqueous NaHCO₃ (10 mL). The resulting mixture was extracted with DCM (20 mL*3). The combined organics were washed with brine (30 mL), dried with anhydrous Na₂SO₄, filtered and concentrated in vacuum to afford 4-[4-[(1S, 4S)-2,5-diazabicyclo[2.2.1]heptan-2-yl]-8-fluoro-2-(1,2,3,5,6,7-hexahydropyrrolizin-8-ylmethoxy)pyrido[4,3-d]pyrimidin-7-yl]-5-(2-triisopropylsilylethynyl)naphthalen-2-ol (180 mg, 71% yield) as a brown solid.

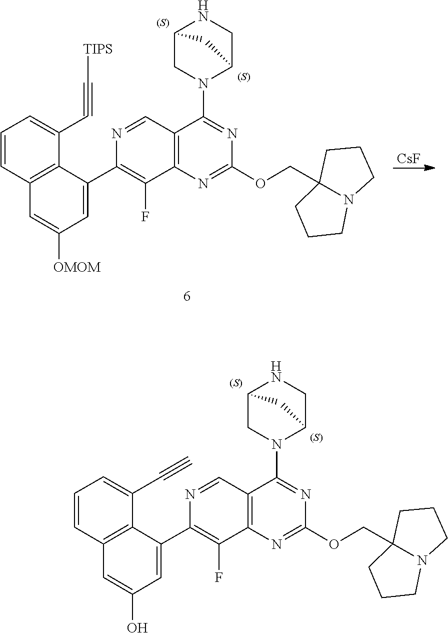

To a solution of 4-[4-[(1S, 4S)-2,5-diazabicyclo[2.2.1]heptan-2-yl]-8-fluoro-2-(1,2,3,5,6,7-hexahydropyrrolizin-8-ylmethoxy)pyrido[4,3-d]pyrimidin-7-yl]-5-(2-triisopropylsilylethynyl) naphthalene-2-ol (180 mg, 0.25 mmol) in DMF (4 mL) was added CsF (387 mg, 2.55 mmol). The mixture was stirred at 25° C. for 12 h. The mixture was filtered and the filtrate was purified by preparative HPLC (column: Waters Xbridge BEH CIS 100*25 mm*5 um; mobile phase: [Heptane-EtOH (0.1% NH₃·H₂O)];B %: 45%-80%, 8 min) to afford 4-(4-((1S,4S)-2,5-diazabicyclo[2.2.1]heptan-2-yl)-8-fluoro-2-((tetrahydro-1H-pyrrolizin-7a(5H)-yl)methoxy)pyrido[4,3-d]pyrimidin-7-yl)-5-ethynylnaphthalen-2-ol (35 mg, 24% yield) as a yellow solid. MS (ESI) m/z=550.62 [M+H]. ¹H NMR (400 MHz, DMSO-d₆): δ=9.10-9.05 (m, 1H), 7.87 (d, J=8.0 Hz, 1H), 7.53-7.38 (m, 2H), 7.36-7.22 (m, 1H), 7.16-6.99 (m, 111), 5.30-5.05 (m, 1H), 4.42-4.16 (m, 1H), 4.07-3.96 (m, 2H), 3.09-2.60 (m, 8H), 2.23-1.98 (m, 1H), 1.68-1.41 (m, 4H), 1.30-1.19 (m, 10H).

### Example 7

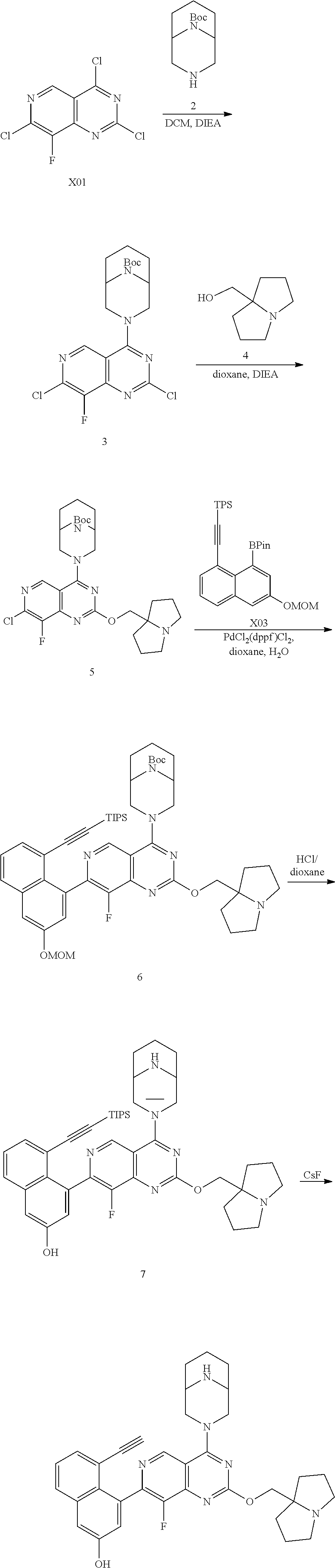

### Procedure for Preparation of Compound 3

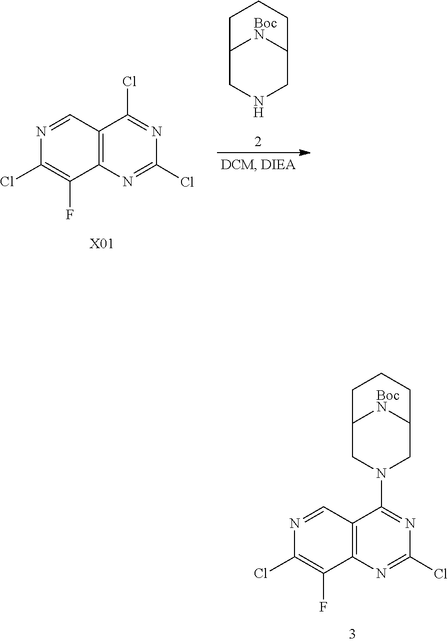

A solution of 2,4,7-trichloro-8-fluoro-pyrido[4,3-d]pyrimidine (500 mg, 1.98 mmol) in DCM (2 mL) was added tert-butyl 3,9-diazabicyclo[3.3.1]nonane-9-carboxylate (448 mg, 1.98 mmol) and DIEA (2.56 g, 19.81 mmol) at −40° C. The mixture was stirred at −40° C. for 0.5 h then concentrated in vacuo to give a residue. The residue was purified by flash column chromatography on silica gel (ethyl acetate in petroleum ether from 0% to 40%) to give tert-butyl 3-(2,7-dichloro-8-fluoro-pyrido[4,3-d]pyrimidin-4-yl)-3,9-diazabicyclo[3.3.1]nonane-9-carboxylate (250 mg, 26% yield) as white solid.

### Procedure for Preparation of Compound 5

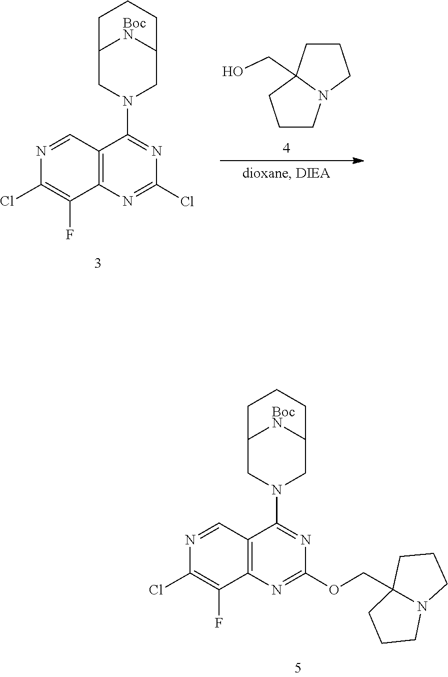 [SMILES: CC(C)(C)OC(=O)N1C2CCCC1CN(c1nc(Cl)nc3c(F)c(Cl)ncc13)C2 ; OCC12CCCN1CCC2 ; CC(C)(C)OC(=O)N1C2CCCC1CN(c1nc(OCC34CCCN3CCC4)nc3c(F)c(Cl)ncc13)C2]

A solution of tert-butyl 3-(2,7-dichloro-8-fluoro-pyrido[4,3-d]pyrimidin-4-yl)-3,9-diazabicyclo[3.3.1]nonane-9-carboxylate (200 mg, 0.452 mmol) and 1,2,3,5,6,7-hexahydropyrrolizin-8-ylmethanol (192 mg, 1.36 mmol) in dioxane (5 mL) was added DIEA (175 mg, 1.36 mmol). The mixture was stirred at 90° C. for 4 h. The mixture was concentrated in vacuo to give a residue. The residue was added water (10 mL), then extracted with EtOAc (10 mL×3). The combined organic layers were washed with H₂O (20 mL×2), dried over Na₂SO₄, filtered and concentrated under reduced pressure to give a residue. The residue was purified by flash column chromatography on silica gel (MeOH in DCM from 0% to 3%) to give tert-butyl 3-[7-chloro-8-fluoro-2-(1,2,3,5,6,7-hexahydropyrrolizin-8-ylmethoxy)pyrido[4,3-d]pyrimidin-4-yl]-3,9-diazabicyclo[3.3.1]nonane-9-carboxylate (80 mg, 28% yield) as red gum.

### Procedure for Preparation of Compound 6

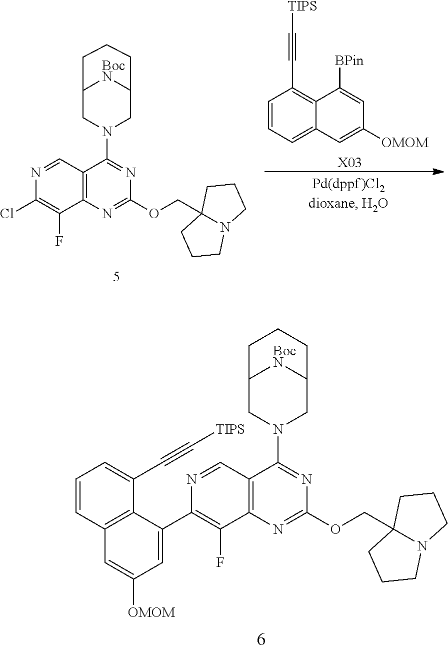

A solution of tert-butyl 3-[7-chloro-8-fluoro-2-(1,2,3,5,6,7-hexahydropyrrolizin-8-ylmethoxy)pyrido[4,3-d]pyrimidin-4-yl]-3,9-diazabicyclo[3.3.1]nonane-9-carboxylate (440 mg, 0.804 mmol) and triisopropyl-[2-[6-(methoxymethoxy)-8-(4,4,5,5-tetramethyl-1,3,2-dioxaborolan-2-yl)-1-naphthyl]ethynyl]silane (398 mg, 0.804 mmol) in dioxane (10 mL) and H₂O (2 mL) was added cesium carbonate (734 mg, 2.25 mmol) and ditert-butyl (cyclopentyl)phosphane;dichloropalladium; iron (105 mg, 0.161 mmol). The mixture was stirred at 100° C. for 2 h at N₂ atmosphere then concentrated in vacuo to give a residue. To the residue was added water (10 mL), then extracted with DCM (15 mL×3). The combined organic layers were washed with H₂O (20 mL×2), dried over Na₂SO₄, filtered and concentrated under reduced pressure to give a residue. The residue was purified by prep-TLC (DCM/MeOH=5/1) to give tert-butyl 3-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-3,9-diazabicyclo[3.3.1]nonane-9-carboxylate (463 mg, 61% yield) as brown oil.

### Procedure for Preparation of Compound 7

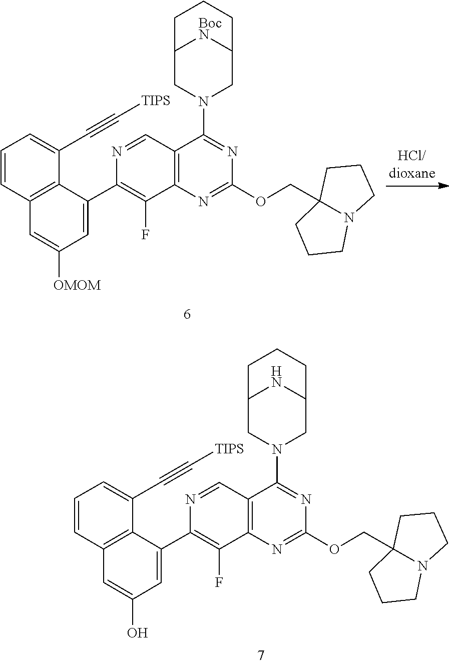

To a mixture of tert-butyl 3-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-3,9-diazabicyclo[3.3.1]nonane-9-carboxylate (150 mg, 0.170 mmol) in dioxane (2 mL) was added HCl (12 M, 0.71 mL) in one portion at 0° C. The mixture was stirred at 20° C. for 1 hour. The mixture was adjusted to pH ˜8 by addition of saturated aqueous NaHCO₃ (10 mL). The resulting mixture was extracted with DCM (20 mL×3). The combined organic phase was washed with brine (30 mL), dried with anhydrous Na₂SO₄, filtered and concentrated in vacuum to afford a residue. The crude product was purified by flash column chromatography on silica gel (methanol in dichloromethane from 0% to 15%) to afford 4-[4-(3,9-diazabicyclo[3.3.1]nonan-3-yl)-8-fluoro-2-(1,2,3,5,6,7-hexahydropyrrolizin-8-ylmethoxy)pyrido[4,3-d]pyrimidin-7-yl]-5-(2-triisopropylsilylethynyl)naphthalen-2-ol (55 mg, 44% yield) as a yellow solid.

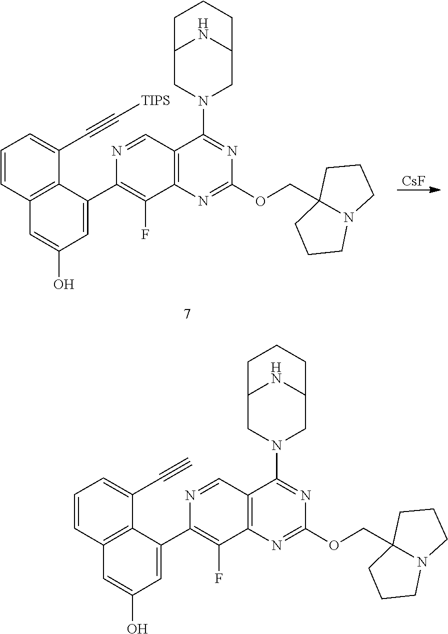

To a mixture of 4-[4-(3,9-diazabicyclo[3.3.1]nonan-3-yl)-8-fluoro-2-(1,2,3,5,6,7-hexahydropyrrolizin-8-ylmethoxy)pyrido[4,3-d]pyrimidin-7-yl]-5-(2-triisopropylsilylethynyl)naphthalen-2-ol (45 mg, 0.061 mmol) in DMF (1 mL) was added CsF (93 mg, 0.612 mmol) in one portion at 25° C. The mixture was stirred at 25° C. for 12 hours. The mixture was filtered and the filtrate was purified by prep-HPLC (column: C18-6 100*30 mm*5 um; mobile phase: [water(HCOONH₄)-ACN]; B %: 8%-43%, 15 min); FlowRate (25 mL/min)) to afford and lyophilized to afford 4-(4-(3,9-diazabicyclo[3.3.1]nonan-3-yl)-8-fluoro-2-((tetrahydro-1H-pyrrolizin-7a(5H)-yl)methoxy)pyrido[4,3-d]pyrimidin-7-yl)-5-ethynylnaphthalen-2-ol (7.6 mg, 21% yield) as a yellow solid. MS (ESI) m/z=579.3 [M+H]⁺. ¹H NMR (400 MHz, DMSO-d₆): δ=9.06 (s, 1H), 7.89-7.87 (m, 1H), 7.46-7.40 (m, 2H), 7.34-7.33 (d, J=2.4 Hz, 1H), 7.14-7.13 (d, J=2.8 Hz, 1H), 4.59-4.55 (m, 2H), 4.09 (s, 4H), 3.56 (s, 2H), 3.23 (s, 3H), 2.99-2.93 (m, 2H), 2.64-2.54 (m, 3H), 1.94-1.92 (m, 1H), 1.87-1.72 (m, 8H), 1.63-1.51 (m, 3H), 1.01-0.98 (t, J=7.2 Hz, 1H).

### Example 8

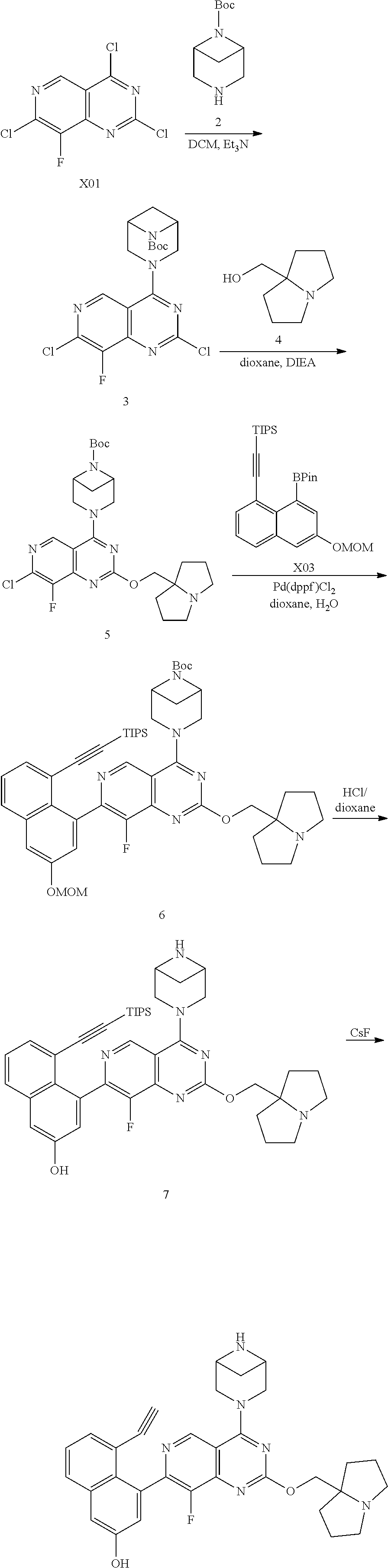

### Procedure for Preparation of Compound 3

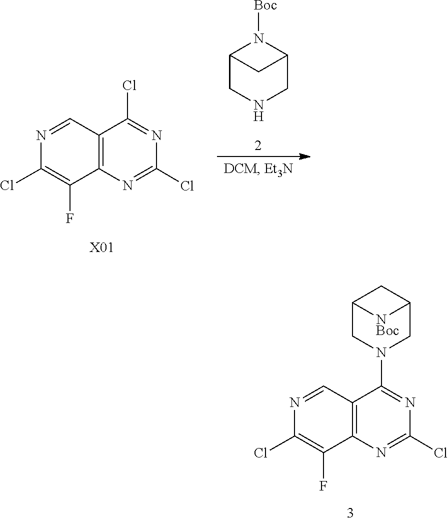

To a solution of 2,4,7-trichloro-8-fluoro-pyrido[4,3-d]pyrimidine (1 g, 3.56 mmol) in DCM (20 mL) was added DIEA (4.61 g, 35 65 mmol) and tert-butyl 3,6-diazabicyclo[3.1.1]heptane-6-carboxylate (707 mg, 3.56 mmol). The mixture was stirred at −40° C. for 0.5 h. The mixture was concentrated in vacuo to give a residue. The residue was purified by flash silica gel chromatography (ISCO@; 12 g SepaFlash® Silica Flash Column, Eluent of 0˜20% petroleum ether/ethyl acetate @60 mL/min) to afford tert-butyl 3-(2,7-dichloro-8-fluoro-pyrido[4,3-d]pyrimidin-4-yl)-3,6-diazabicyclo[3.1.1]heptane-6-carboxylate (620 mg, 33% yield) as a yellow oil.

### Procedure for Preparation of Compound 5

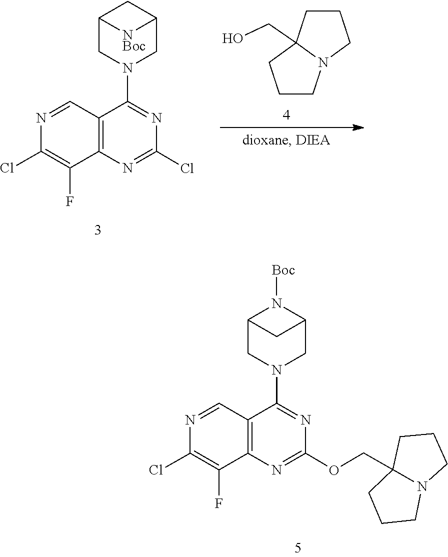 [SMILES: CC(C)(C)OC(=O)N1C2CC1CN(c1nc(Cl)nc3c(F)c(Cl)ncc13)C2 ; OCC12CCCN1CCC2 ; CC(C)(C)OC(=O)N1C2CC1CN(c1nc(OCC34CCCN3CCC4)nc3c(F)c(Cl)ncc13)C2]

To a solution of tert-butyl 3-(2,7-dichloro-8-fluoro-pyrido[4,3-d]pyrimidin-4-yl)-3,6-diazabicyclo[3.1.1]heptane-6-carboxylate (620 mg, 1.50 mmol) in dioxane (20 mL) was added DIEA (580 mg, 4.49 mmol) and 1,2,3,5,6,7-hexahydropyrrolizin-8-ylmethanol (634 mg, 4.49 mmol). The mixture was stirred at 90° C. for 16 h. The mixture was concentrated in vacuo to give a residue. The residue was purified by flash silica gel chromatography (ISCO®; 12 g SepaFlash® Silica Flash Column, Eluent of 0˜10% methanol/dichloromethane @60 mL/min) to afford tert-butyl 3-[7-chloro-8-fluoro-2-(1,2,3,5,6,7-hexahydropyrrolizin-8-ylmethoxy)pyrido[4,3-d]pyrimidin-4-yl]-3,6-diazabicyclo[3.1.1]heptane-6-carboxylate (960 mg, 98% yield) as a yellow solid.

### Procedure for Preparation of Compound 6

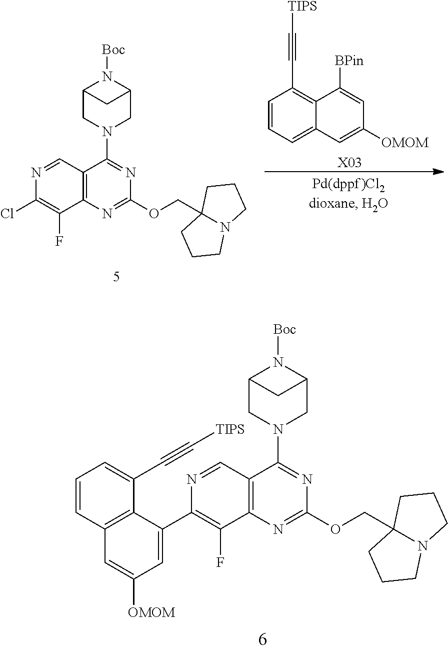

To a mixture of triisopropyl-[2-[6-(methoxymethoxy)-8-(4,4,5,5-tetramethyl-1,3,2-dioxaborolan-2-yl)-1-naphthyl]ethynyl]silane (438 mg, 0.886 mmol) and tert-butyl 3-[7-chloro-8-fluoro-2-(1,2,3,5,6,7-hexahydropyrrolizin-8-ylmethoxy)pyrido[4,3-d]pyrimidin-4-yl]-3,6-diazabicyclo[3.1.1]heptane-6-carboxylate (400 mg, 0.770 mmol) in dioxane (15 mL) and H₂O (3 mL) was added Cs₂CO₃ (703 mg, 2.16 mmol) and ditertbutyl(cyclopentyl)phosphane;dichloropalladium;iron (100 mg, 0.154 mmol) in one portion at 25° C. under N₂. The mixture was stirred at 100° C. for 16 h then extracted with ethyl acetate (10 mL×3) and concentrated. The residue was purified by flash silica gel chromatography (ISCO®; 4 g SepaFlash® Silica Flash Column, eluent of 0˜8% Dichloromethane/Methanol @40 mL/min) to afford tert-butyl 3-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-3,6-diazabicyclo[3.1.1]heptane-6-carboxylate (192 mg, 27% yield) as a brown solid.

### Procedure for Preparation of Compound 7

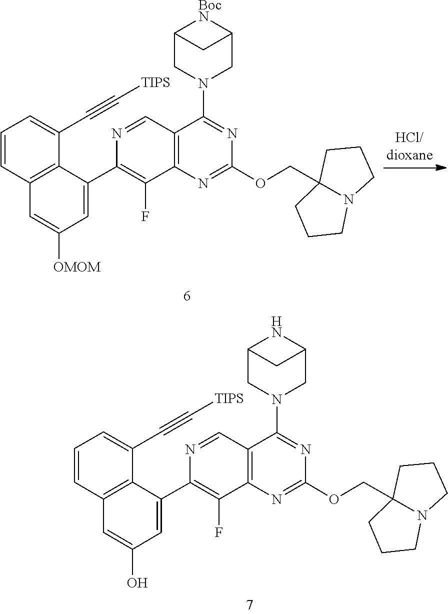

To a solution of tert-butyl 3-[8-fluoro-2-(1,2,3,5,6,7-hexahydropyrrolizin-8-ylmethoxy)-7-[3-(methoxymethoxy)-8-(2-triisopropylsilylethynyl)-1-naphthyl]pyrido[4,3-d]pyrimidin-4-yl]-3,6-diazabicyclo[3.1.1]heptane-6-carboxylate (100 mg, 0.117 mmol) in dioxane (1 mL) was added HCl (12 M, 0.49 mL) at 0° C. The mixture was stirred at 25° C. for 1 h then adjusted to pH˜8 by addition of saturated aqueous NaHCO₃ (10 mL). The resulting mixture was extracted with DCM (20 mL×3). The combined organic phase was washed with brine (30 mL), dried with anhydrous Na₂SO₄, filtered and concentrated in vacuum to afford 4-[4-(3,6-diazabicyclo[3.1.1]heptan-3-yl)-8-fluoro-2-(1,2,3,5,6,7-hexahydropyrrolizin-8-ylmethoxy)pyrido[4,3-d]pyrimidin-7-yl]-5-(2-triisopropylsilylethynyl)naphthalen-2-ol (80 mg, 63% yield) as a yellow solid.

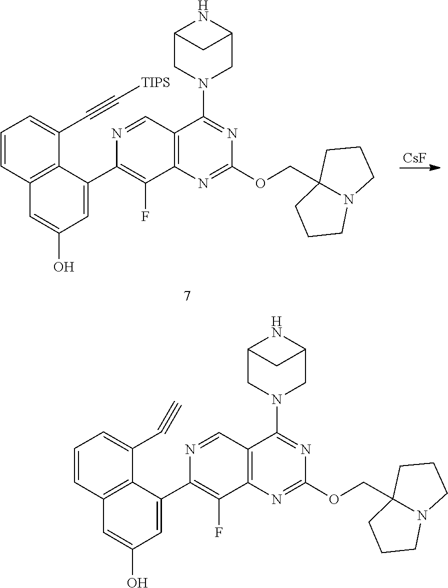

To a mixture of 4-[4-(3,6-diazabicyclo[3.1.1]heptan-3-yl)-8-fluoro-2-(1,2,3,5,6,7-hexahydropyrrolizin-8-ylmethoxy)pyrido[4,3-d]pyrimidin-7-yl]-5-(2-triisopropylsilylethynyl)naphthalen-2-ol (80 mg, 0.113 mmol) in DMF (2 mL) was added CsF (171.89 mg, 1.13 mmol) in one portion at 25° C. The mixture was stirred at 25° C. for 12 hours. The mixture was filtered and the filtrate was purified by prep-HPLC (column: C18-6 100*30 mm*5 um; mobile phase: [water (FA)-ACN]; B %: 0%-40%, 15 min); flowRate (25 mL/min)) to afford 4-[4-(3,6-diazabicyclo[3.1.1]heptan-3-yl)-8-fluoro-2-(1,2,3,5,6,7-hexahydropyrrolizin-8-ylmethoxy)pyrido[4,3-d]pyrimidin-7-yl]-5-ethynyl-naphthalen-2-ol (10.3 mg, 17% yield) as a yellow solid. MS (ESI) m/z=551.3 [M+H]⁺. ¹H NMR (400 MHz, DMSO-d₆): δ=9.41 (s, 1H), 8.24 (s, 2H), 7.89-7.87 (d, J=7.6 Hz, 1H), 7.47-7.40 (m, 2H), 7.34 (s, 1H), 7.13 (s, 1H), 4.37 (m, 1H), 4.27-4.22 (m, 4H), 3.95 (s, 2H), 3.12-3.09 (m, 2H), 2.91-2.85 (m, 1H), 2.74-2.67 (m, 3H), 1.98-1.80 (m, 6H), 1.73-1.64 (m, 2H), 1.23-0.85 (m, 2H).

### Example 9

### Synthesis of X01

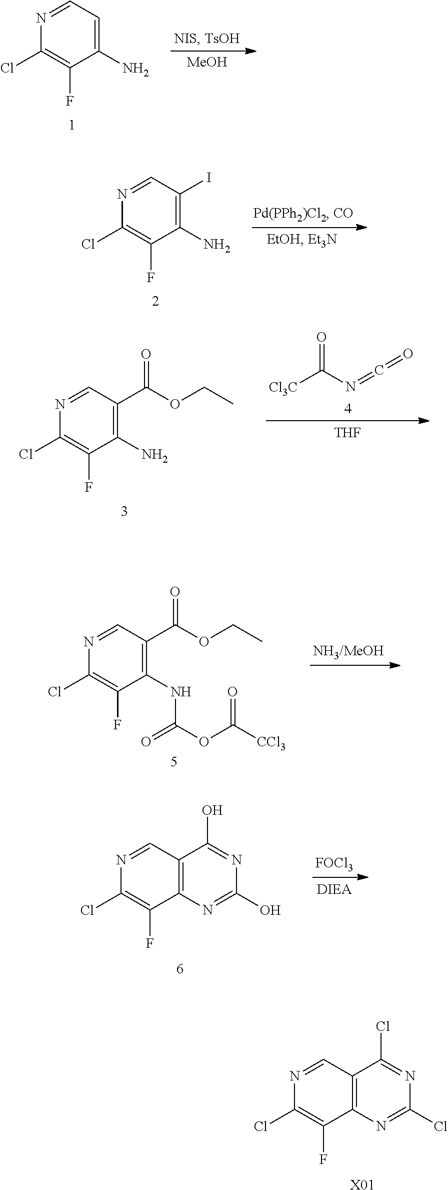

### Procedure for Preparation of Compound 2

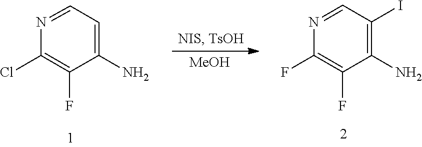 [SMILES: "MeOH" ; Nc1ccnc(Cl)c1F ; Nc1c(I)cnc(F)c1F]

To a solution of 2-chloro-3-fluoro-pyridin-4-amine (41.8 g, 285.23 mmol), NIS (77.01 g, 342.27 mmol) and NIS (77.01 g, 342.27 mmol) in MeCN (560 mL) was added TsOH·H₂O (2.71 g, 14.26 mmol). The mixture was stirred at 70° C. for 16 h. The mixture was diluted with water (500 mL) and extracted with ethyl acetate (500 mL×3). The organic layer was washed with statured Na₂CO₃ solution (500 mL×2), saturated Na₂SO₃ (500 ml) solution and brine (80 ml), dried over Na₂SO₄, filtered and concentrated under vacuum afford 2-chloro-3-fluoro-5-iodo-pyridin-4-amine (65.64 g, 70% yield) as a yellow solid.

### Procedure for Preparation of Compound 3

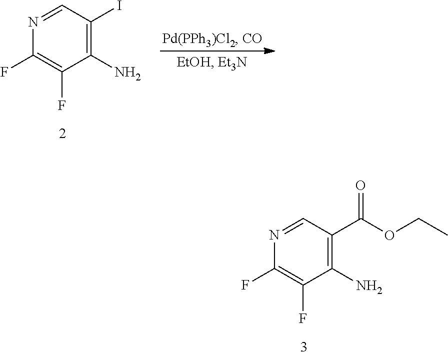

A mixture of 2-chloro-3-fluoro-5-iodo-pyridin-4-amine (65.64 g, 240.93 mmol), Pd(PPh₃)₂Cl₂ (16.91 g, 24.09 mmol) in EtOH (820 mL) and Et₃N (73 g, 723 mmol) was degassed and purged with N₂ for 3 times, and then the mixture was stirred at 80° C. for 15 h under CO (15 psi). The mixture was concentrated to dryness, then the residue was purified by flash column chromatography on silica gel (DCM in PE from 0% to 30%) to afford ethyl 4-amino-6-chloro-5-fluoro-pyridine-3-carboxylate (13.71 g, 26% yield) as a yellow solid.

### Procedure for Preparation of Compound 5

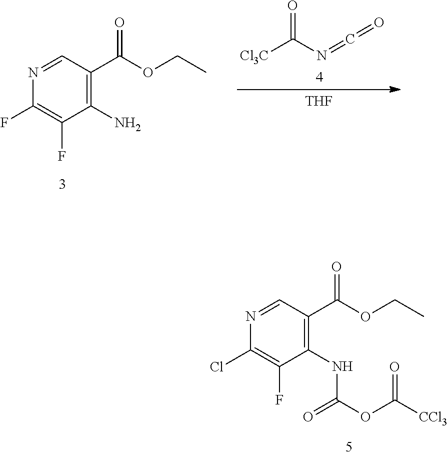

To a solution of ethyl 4-amino-6-chloro-5-fluoro-pyridine-3-carboxylate (17 g, 77.76 mmol) in THE (40 mL) was added 2,2,2-trichloroacetyl isocyanate (21.98 g, 116.64 mmol). The mixture was stirred at 25° C. for 10 min. Upon completion, the mixture was concentrated under vacuum, the crude product was triturated with MTBE (100 mL) at 25° C. for 5 min and filtered. The filter cake was washed with MTBE (30 mL×2), dried to afford ethyl 6-chloro-5-fluoro-4-[(2,2,2-trichloroacetyl)oxycarbonylamino]pyridine-3-carboxylate (20 g, 51% yield) as a gray solid.

### Procedure for Preparation of Compound 6

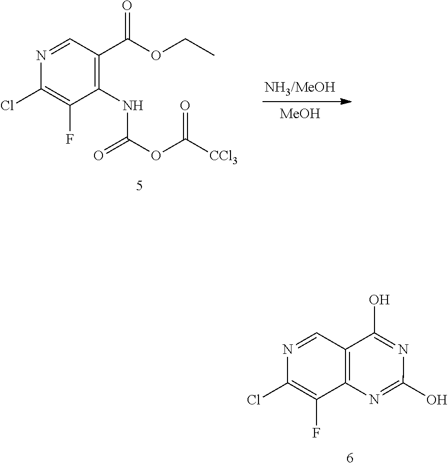

To a solution of ethyl 6-chloro-5-fluoro-4-[(2,2,2-trichloroacetyl)oxycarbonylamino]pyridine-3-carboxylate (20 g, 49.02 mmol) in MeOH (150 mL) was added NH₃·H₂O (76.50 g, 4.49 mol). The mixture was stirred at 25° C. for 1 h. The mixture was concentrated under vacuum. The crude product was triturated with MTBE (80 mL) at 25° C. for 10 min and the residue was filtered. The filter cake was washed with MTBE (30 mL×2), dried to afford 7-chloro-8-fluoro-pyrido [4,3-d]pyrimidine-2,4-diol (11.74 g, crude) as a white solid.

### Procedure for Preparation of Compound X01

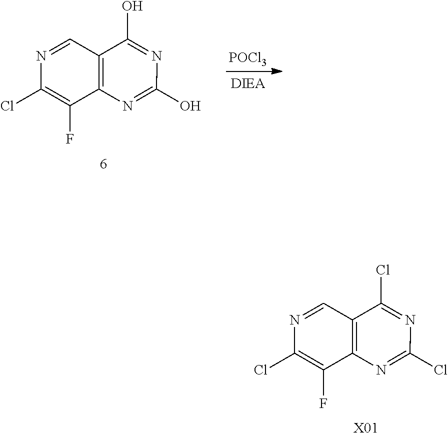

To a solution of POCl₃ (82.50 g, 538.06 mmol) in DIEA (14.99 g, 115.97 mmol) was added 7-chloro-8-fluoro-pyrido[4,3-d]pyrimidine-2,4-diol (5 g, 23.19 mmol) at 0° C. then the mixture was stirred at 100° C. for 2 h. The mixture was concentrated under reduced pressure. To the residue was added ice water (60 mL) then it was filtered, and the filter cake was washed with MTBE (20 mL×2) and dried to afford 2,4,7-trichloro-8-fluoro-pyrido[4,3-d]pyrimidine (4.38 g, 17.33 mmol) as a yellow solid.

### Example 10

### Synthesis of X03

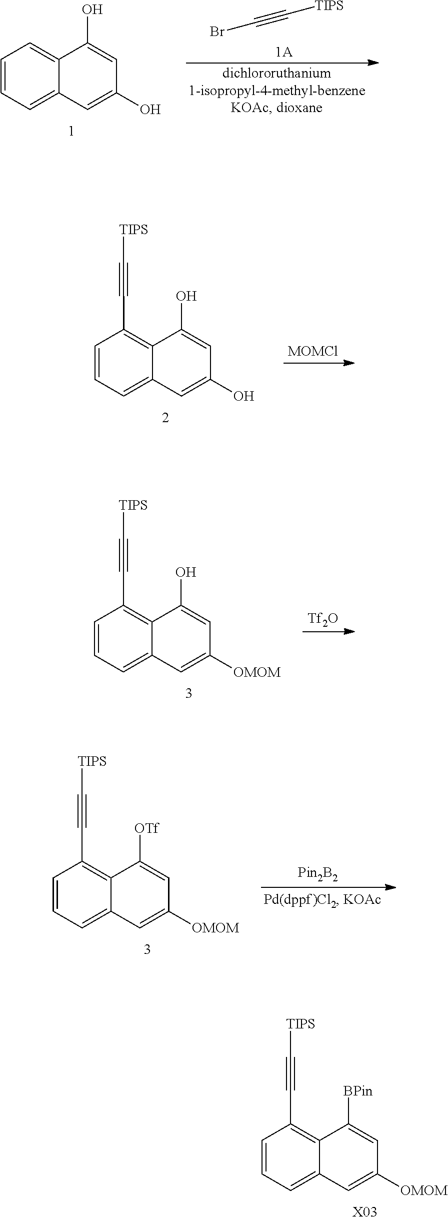

### Procedure for Preparation of Compound 2

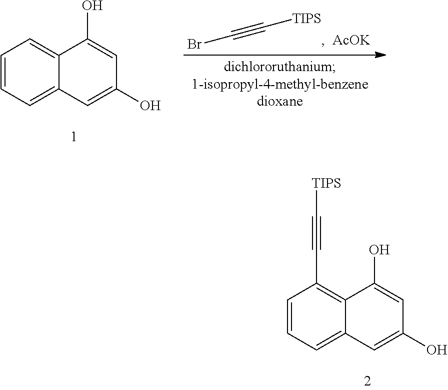

A mixture of naphthalene-1,3-diol (6.5 g, 40.58 mmol), 2-bromoethynyl(triisopropyl)silane (12.72 g, 48.70 mmol), AcOK (7.97 g, 81.16 mmol), dichlororuthenium;1-isopropyl-4-methyl-benzene (2.49 g, 4.06 mmol) in dioxane (150 mL) was degassed and purged with N₂ for 3 times, and then the mixture was stirred at 110° C. for 12 h under N₂ atmosphere. The mixture was partitioned between H₂O (80 mL) and ethyl acetate (80 mL). The organic phase was separated, and the aqueous phase was extracted with ethyl acetate (80 mL×2). The combine organic layer was washed with brine (80 mL), dried over Na₂SO₄, filtered and concentrated under vacuum. The residue was purified by flash column chromatography on silica gel (ethyl acetate in petroleum ether from 0% to 2%) to afford 8-(2-triisopropylsilylethynyl) naphthalene-1,3-diol (8.88 g, 61% yield) as a brown solid.

### Procedure for Preparation of Compound 3

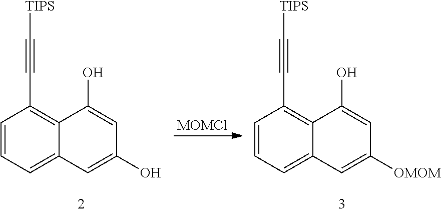

To a solution of 8-(2-triisopropylsilylethynyl)naphthalene-1,3-diol (8.88 g, 26.08 mmol) in DCM (100 mL) was added DIEA (10.11 g, 78.23 mmol) and MOMCl (3.15 g, 39.12 mmol) at 0° C. The mixture was stirred at 0° C. for 0.5 h. The mixture was diluted with water (100 mL) and separated. The water phase was extracted with dichloromethane (150 mL) and the combined organic layer was washed with brine (100 mL), dried over Na₂SO₄, filtered and concentrated under vacuum. The residue was purified by flash column chromatography on silica gel (EtOAc in PE from 0% to 2%) to afford 3-(methoxymethoxy)-8-(2-triisopropylsilylethynyl) naphthalen-1-ol (6.4 g, 62% yield) as a yellow solid.

### Procedure for Preparation of Compound 4

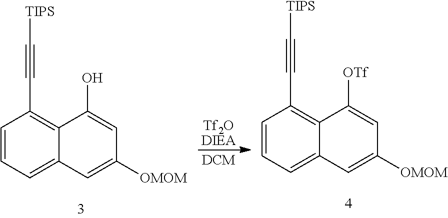

To a solution of 3-(methoxymethoxy)-8-(2-triisopropylsilylethynyl) naphthalen-1-ol (6.4 g, 16.64 mmol) in CH₂Cl₂ (70 mL) was added DIEA (6.45 g, 49.92 mmol) and Tf₂O (7.04 g, 24.96 mmol) at −40° C. The mixture was stirred at −40° C. for 0.5 h then quenched by addition of water (120 mL) at −40° C., the water phase was extracted with dichloromethane (30 mL). The combined organics were washed with brine (60 mL), dried over Na₂SO₄, filtered and concentrated under vacuum. The residue was purified by flash column chromatography on silica gel (DCM in PE from 0% to 1%) to afford [3-(methoxymethoxy)-8-(2-triisopropylsilylethynyl)-1-naphthyl]trifluoromethanesulfonate (12.0 g, crude) as a yellow oil. MS (ESI) m/z=517.2 [M+H]⁺. ¹H NMR (400 MHz, CDCl₃): δ=1.14-1.27 (m, 21H) 3.52 (d, J=1.8 Hz, 3H) 5.29 (d, J=1.8 Hz, 2H) 7.31 (s, 1H) 7.39-7.51 (m, 2H) 7.68-7.81 (m, 2H).

### Procedure for Preparation of X03

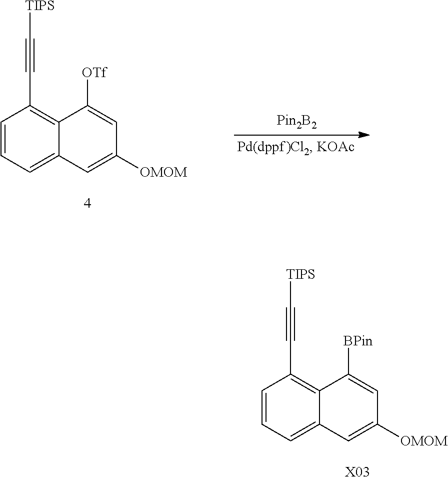

A mixture of [3-(methoxymethoxy)-8-(2-triisopropylsilylethynyl)-1-naphthyl]trifluoromethanesulfonate (12 g, 23.23 mmol), 4,4,5,5-tetramethyl-2-(4,4,5,5-tetramethyl-1,3,2-dioxaborolan-2-yl)-1,3,2-dioxaborolane (29.49 g, 116.13 mmol), Pd(dppf)Cl₂ (1.70 g, 2.32 mmol), KOAc (7.98 g, 81.29 mmol) in toluene (500 mL) was degassed and purged with N₂ 3 times, and then the mixture was stirred at 110° C. for 12 h under N₂ atmosphere. The mixture was concentrated under reduced pressure and the residue was purified by flash column chromatography on silica gel (ethyl acetate in petroleum ether from 0% to 2%) to afford. The product of triisopropyl-[2-[6-(methoxymethoxy)-8-(4,4,5,5-tetramethyl-1,3,2-dioxaborolan-2-yl)-1-naphthyl]ethynyl]silane (5.52 g, 47% yield) as a yellow solid. ¹H NMR (400 MHz, CDCl₃): δ=7.71-7.68 (m, 2H), 7.47 (d, J=2.4 Hz, 1H), 7.38-7.33 (m, 2H), 5.29 (s, 2H), 3.51 (s, 3H), 1.44 (s, 12H), 1.16 (s, 21H).

### Example 11

### HTRF-Based Nucleotide Exchange Assay Detecting GTP Binding to K-Ras

A HTRF-based nucleotide exchange assay is used to characterize the pharmacological properties of the synthesized inhibitor compounds and the IC₅₀ measurement is performed as described below. Kras G12D was mixed with a-GST Tb antibody (1.5× solution) and 10 uL was delivered to reaction wells. A mixture of Kras G12D/aGST-Tb Ab/compound mixture was incubated for 1 h at RT. A 3× solution of SOS1 (aa 564-1049) and fluorescently labelled GTP* was prepared in reaction buffer and 5 μL of SOS1/GTP* solution was added to reaction well to initiate the exchange reaction. An IC₅₀ was determined using a sigmoidal dose response (variable slope) equation. Note: Assay IC₅₀ data are designated within the following ranges: A: ≤10 nM, B: >10 nM to ≤1000 nM, C: >1000 nM

| Example | | KRAS HTRF: |
|---|---|---|
| Number | Structure | IC₅₀ (nM) |
| 1 | 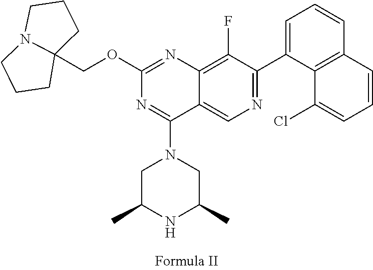 [SMILES: C[C@@H]1CN(c2nc(OCC34CCCN3CCC4)nc3c(F)c(-c4cccc5cccc(Cl)c45)ncc23)C[C@H](C)N1] | C |
| 2 | 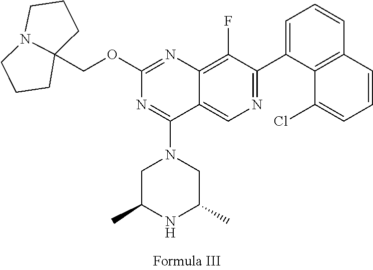 [SMILES: C[C@H]1CN(c2nc(OCC34CCCN3CCC4)nc3c(F)c(-c4cccc5cccc(Cl)c45)ncc23)C[C@H](C)N1] | B |
| 3 | 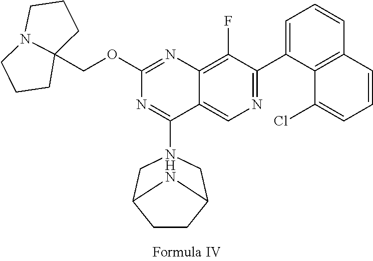 | A |
| 4 | 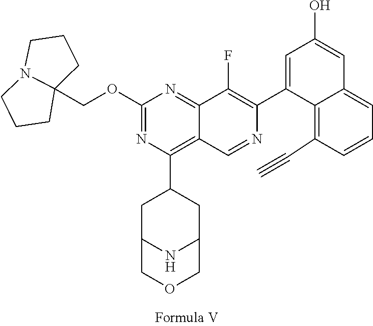 [SMILES: C#Cc1cccc2cc(O)cc(-c3ncc4c(C5CC6COCC(C5)N6)nc(OCC56CCCN5CCC6)nc4c3F)c12] | A |
| 5 | 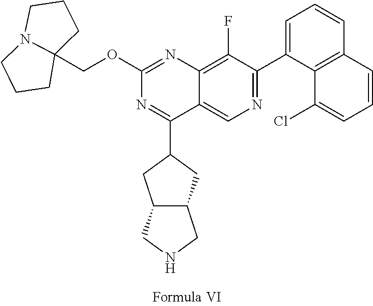 | C |
| 6 | 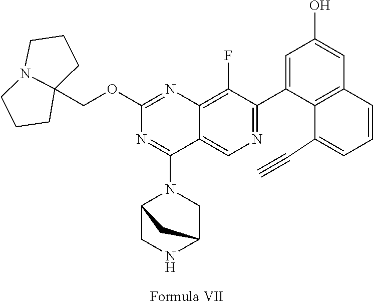 [SMILES: C#Cc1cccc2cc(O)cc(-c3ncc4c(N5C[C@H]6C[C@@H]5CN6)nc(OCC56CCCN5CCC6)nc4c3F)c12] | A |
| 7 | 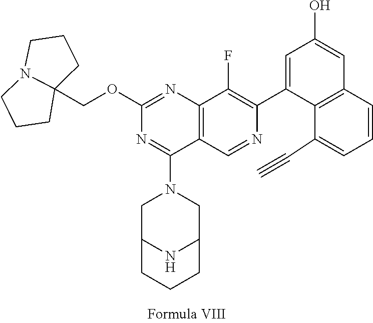 [SMILES: C#Cc1cccc2cc(O)cc(-c3ncc4c(N5CC6CCCC(C5)N6)nc(OCC56CCCN5CCC6)nc4c3F)c12] | A |
| 8 | 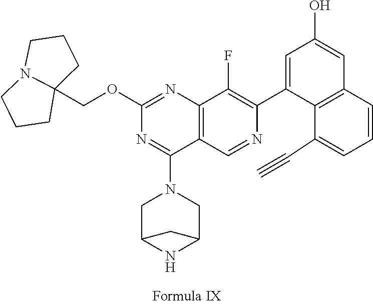 [SMILES: C#Cc1cccc2cc(O)cc(-c3ncc4c(N5CC6CC(C5)N6)nc(OCC56CCCN5CCC6)nc4c3F)c12] | A |

Those skilled in the art will recognize, or be able to ascertain, using no more than routine experimentation, numerous equivalents to the specific composition and procedures described herein. Such equivalents are considered to be within the scope of this disclosure, and are covered by the following claims.

What is claimed is:

1. A compound according to Formula (I) 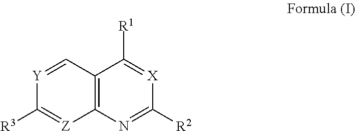
or an optically pure stereoisomer, pharmaceutically acceptable salt, or solvate thereof, wherein
X is a nitrogen or an unsubstituted C₁ group;
Y is a nitrogen, a substituted or an unsubstituted C₁ group;
Z is a nitrogen, a substituted or an unsubstituted C₁ group;
R¹ is selected from the group consisting of: 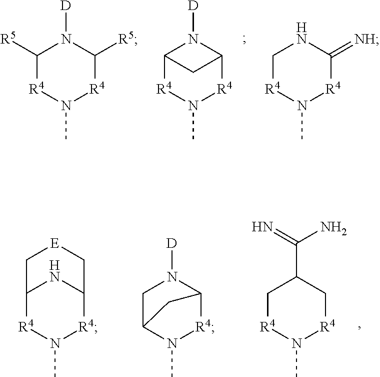
wherein R⁴ is independently in each instance a bond or a C₁₋₂ alkyl group;
R⁵ is independently in each instance a hydrogen or a C₁ alkyl group;
D is a hydrogen or a C₁ alkyl;
E is a NH, oxygen, carbonyl, or substituted or unsubstituted C₁ alkyl;
R² is selected from the group consisting of: 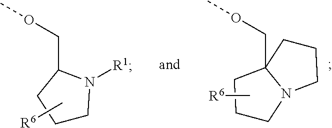
wherein R⁶ is independently selected from the group consisting of a hydrogen, a halogen, and an alkyl group; and
R³ is a substituted or unsubstituted C₆₋₁₈ aryl group.

2. The compound of claim 1 wherein E is selected from the group consisting of NH, O, CH₂, CHOH, CH₂F, CHF₂, and C═O.

3. The compound of claim 1 wherein R⁶ is a C₁₋₅ alkyl group.

4. The compound of claim 1 wherein R³ is a substituted or unsubstituted C₅₋₆ aryl group, a C₁₀₋₁₂ biaryl group, or a C₅₋₆ heteroaryl group, wherein if substituted, substitution is selected from the group consisting of alkyl, —OMe, —F, —Cl, and —OMe alone or with di- or tri-substitutions.

5. The compound of claim 1 wherein Y and Z are independently at each instance a substituted C₁ alkyl group.

6. The compound of claim 5 wherein Y and Z are independently at each instance a CF, or a CCl group.

7. The compound of claim 1 wherein R² is

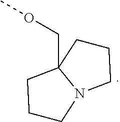

8. The compound of claim 1 wherein R³ is a monosubstituted or a disubstituted naphthyl group.

9. The compound of claim 8 wherein R³ is selected from the group consisting of:

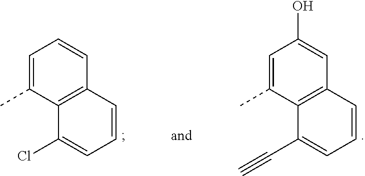

10. The compound of claim 1 having formula X: 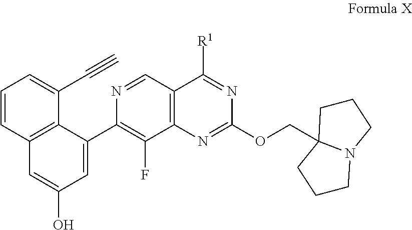
wherein R¹ is selected from the group consisting of:

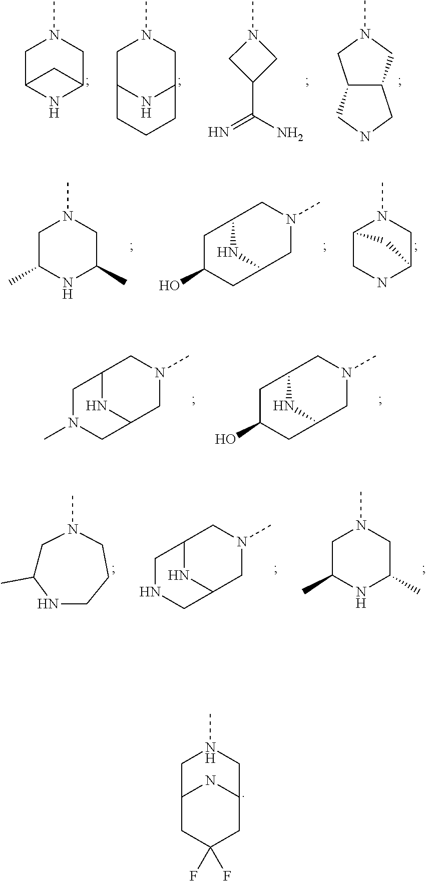

11. The compound of claim 1 being selected from the group consisting of the following compounds:

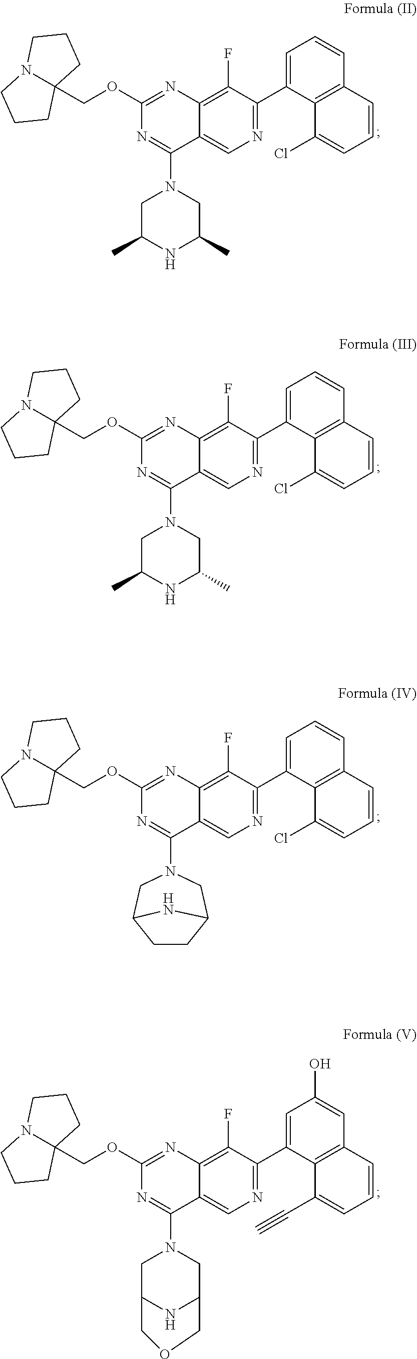 [SMILES: C[C@@H]1CN(c2nc(OCC34CCCN3CCC4)nc3c(F)c(-c4cccc5cccc(Cl)c45)ncc23)C[C@H](C)N1 ; C[C@H]1CN(c2nc(OCC34CCCN3CCC4)nc3c(F)c(-c4cccc5cccc(Cl)c45)ncc23)C[C@H](C)N1 ; Fc1c(-c2cccc3cccc(Cl)c23)ncc2c(N3CC4CCC(C3)N4)nc(OCC34CCCN3CCC4)nc12 ; C#Cc1cccc2cc(O)cc(-c3ncc4c(N5CC6COCC(C5)N6)nc(OCC56CCCN5CCC6)nc4c3F)c12]

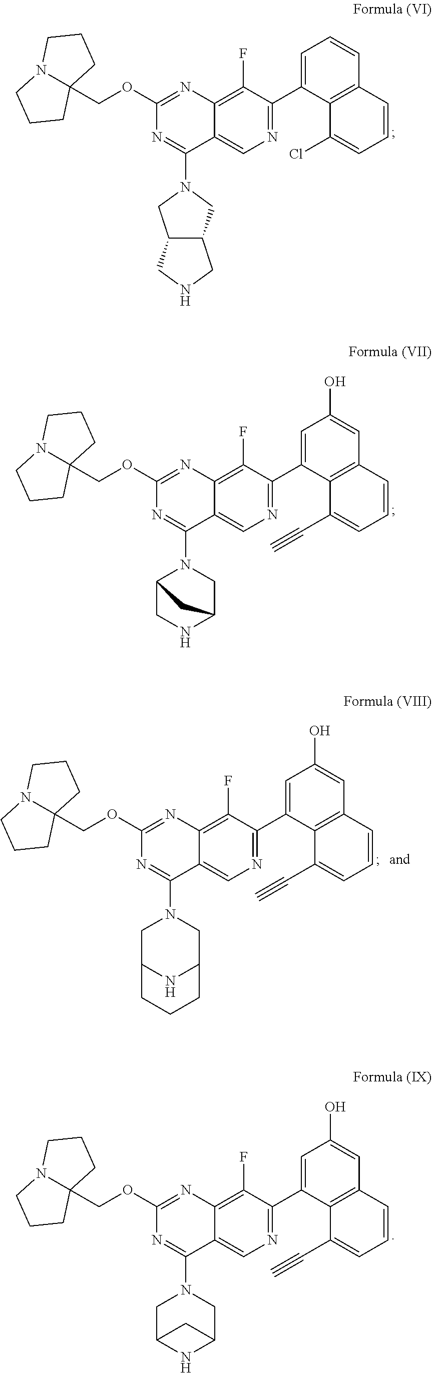 [SMILES: Fc1c(-c2cccc3cccc(Cl)c23)ncc2c(N3C[C@H]4CNC[C@H]4C3)nc(OCC34CCCN3CCC4)nc12 ; C#Cc1cccc2cc(O)cc(-c3ncc4c(N5C[C@H]6C[C@@H]5CN6)nc(OCC56CCCN5CCC6)nc4c3F)c12 ; C#Cc1cccc2cc(O)cc(-c3ncc4c(N5CC6CCCC(C5)N6)nc(OCC56CCCN5CCC6)nc4c3F)c12 ; C#Cc1cccc2cc(O)cc(-c3ncc4c(N5CC6CC(C5)N6)nc(OCC56CCCN5CCC6)nc4c3F)c12]

12. The compound of claim 1, wherein the compound is a GTPase inhibitor.

13. A pharmaceutical composition comprising the compound of claim 1 and a pharmaceutically acceptable carrier.

14. A method of treating cancer in a subject comprising administering to the subject the composition of claim 13 in a therapeutically effective amount.

15. The method of claim 14, wherein the cancer is selected from the group consisting of breast, myeloid, lung, bladder, prostate, ovarian, endometrial, rhabdomyosarcoma, liver, gastric, intestinal, colon/colorectal, and pancreatic cancers.
